(19) United States
(12) Patent Application Publication
(10) Pub. No.: US 2019/0117078 A1
(43) Pub. Date: Apr. 25, 2019

(54) OPTICAL SYSTEMS AND METHODS FOR EXAMINING A TOOTH

(71) Applicant: SONENDO, INC., Laguna Hills, CA (US)

(72) Inventors: Manu Sharma, Ladera Ranch, CA (US); Mehrzad Khakpour, Laguna Hills, CA (US); Bjarne Bergheim, Mission Viejo, CA (US)

(21) Appl. No.: 16/129,529

(22) Filed: Sep. 12, 2018

Related U.S. Application Data
(60) Provisional application No. 62/557,648, filed on Sep. 12, 2017.
(60) Provisional application No. 62/569,260, filed on Oct. 6, 2017.
(60) Provisional application No. 62/570,037, filed on Oct. 9, 2017.
(60) Provisional application No. 62/571,081, filed on Oct. 11, 2017.
(60) Provisional application No. 62/584,638, filed on Nov. 10, 2017.

(51) Int. Cl. A61B 5/00; A61B 5/01; A61B 1/24; A61B 1/07

(52) U.S. Cl. CPC A61B 5/0088; A61B 5/0071; A61B 5/0066; A61B 5/0075; A61B 1/24; A61B 1/07; A61B 5/015

(57) ABSTRACT

Systems and method for optical examination of dental structures are disclosed. Light from an optical source is directed on a portion of a dental structure. Light received from the portion of the dental structure is analyzed to detect a condition of the dental structure.

### CROSS-REFERENCE TO RELATED APPLICATIONS

This application claims the benefit of U.S. Provisional Application No. 62/557,648, filed on Sep. 12, 2017; U.S. Provisional Application No. 62/569,260, filed on Oct. 6, 2017; U.S. Provisional Application No. 62/570,037, filed on Oct. 9, 2017; U.S. Provisional Application No. 62/571,081, filed on Oct. 11, 2017; U.S. Provisional Application No. 62/584,638, filed on Nov. 10, 2017. Each of the above referenced application is incorporated herein by reference in their entireties.

### BACKGROUND

### Field

This disclosure generally relates to optical systems and methods to detect caries, cracks, tooth defects and oral pathologies.

### Description of Related Art

Dental caries, also known as tooth decay or a cavity, is one of the most common chronic diseases in the world. Caries is an infection that causes demineralization of the hard tissues (e.g., enamel, dentin and cementum) and destruction of the organic matter of the tooth, often by production of acid by hydrolysis of the food debris accumulated on the tooth surface. If demineralization exceeds remineralization from saliva, or from other factors such as the use of calcium and fluoridated toothpastes, these tissues may progressively break down, producing dental caries (e.g., cavities or holes in the teeth). If left untreated, the disease can lead to pain, tooth loss and infection. While caries may be directly visible, the caries and its extent of destruction may be detected and evaluated by imaging, e.g. radiographs, as well as by tactile inspection. Caries may form and develop anywhere on the tooth, e.g., occlusal surfaces (pits and fissure caries), proximal and cervical surfaces (smooth surface caries), root surfaces, etc.

Caries or cavities may progress in various stages. For example, early stage caries may be non-cavitated, in which decay has progressed within the enamel, but not below the enamel into dentin. If the caries do not progress any further, then no or minimal treatment may be adequate. However, if there is further progression into the enamel, then treatments, such as the application of a sealant and/or antimicrobial or fluoride agents, may be desirable. If the decay progresses below the enamel and into the dentin, but not reaching the pulp, then a clinician can treat the tooth by restoring the tooth and applying antimicrobial and/or fluoride agents. For caries that progress into the pulpal cavity, endodontic treatment is often advised. Endodontic treatement can include removal of inflamed or infected pulp, cleaning and/or disinfection of the pulpal cavity followed by filling and sealing of a rubber-like material.

Dental caries generally contain bacteria and their byproducts, food remnants, healthy tissue and decayed tissue, and may include other organic and/or inorganic materials. Organic material (or organic matter) includes organic substances typically found in healthy or diseased teeth or root canal systems such as, for example, soft tissue, blood vessels, nerves, connective tissue, cellular matter, pus, microorganisms, bacteria, biofilms, and plaque, whether living, inflamed, infected, diseased, necrotic, or decomposed. Inorganic matter includes calcified tissue and calcified structures, calculus, tartar, etc., which are frequently present in or on teeth.

Non-invasive and non-destructive methods of examining a tooth and/or the pulpal cavity can be advantageous to detect caries, cavities, cracks and/or determine concentration of bacteria in the pulpal cavity.

### SUMMARY

Embodiments described herein have several features, no single one of which is solely responsible for their desirable attributes. Without limiting the scope of the invention as expressed by the claims, some of the advantageous features will now be discussed briefly.

Optical techniques utilizing the differences between the interaction of electromagnetic radiation with different types of biological tissue can be used to obtain information about the presence of diseases and precursors to diseases. This application contemplates using various optical techniques to identify diseases, defects and/or morphological changes in various dental tissues. The optical techniques contemplated in this application can be used to obtain information regarding efficacy of a treatment. For example, the optical techniques contemplated in this application can be used to compare pre-treatment and post-treatment conditions of various dental tissues. The optical techniques contemplated in this application can be used to the etiology of various diseases affecting dental tissue. This application also contemplates determining one or more characteristics representative of the dental tissue based on an analysis of the information obtained by the optical techniques described in this application. Additionally, this application contemplates a system (e.g., an automated electronic system) that can detect caries, crack, cavities and/or changes to the morphology of a dental tissue based on an analysis of the information obtained by the optical techniques described in this application. Furthermore, this application contemplates a system (e.g., an automated electronic system) that can provide clinical feedback prior to or during an endodontic treatment procedure. This application also contemplates a visualization system that can display one or more characteristics representative of a defect, a change in the morphology of the tooth and/or an amount of bacteria in different portions of the tooth.

Various embodiments of optical systems and methods described herein provide the ability to optically interrogate root canals, carious lesions, cracks in enamel of the tooth and/or periodontal pathology. Various embodiments described herein comprise optical systems and methods that can be positioned to direct light onto one or more teeth of a patient and receive light emitted by, reflected from or scattered by different portions of the one or more teeth of the patient. The received light emitted by, reflected from or scattered by different portions of the one or more teeth of the patient can be used to detect caries and tooth defects (e.g., cracks), estimate the amount of bacteria in the root canal of the one or more teeth of the patient, measure the canal working length, characterize the structure and architecture of inner canal wall structure of the one or more teeth, visualize and diagnose periodontium pathologies and/or visualize apical region of a root canal.

This application contemplates a device capable of measuring the depth and bacterial load of a periodontal pocket. This application contemplates a device capable of detecting and imaging caries on a sequence of teeth, with visual display for the user by color-coding the tooth surface according to pathological, metabolic or morphological state. This application contemplates a device capable of detecting cracks. This application contemplates a device capable of measuring root canal working length. This application contemplates a device capable of providing cross-sectional images of root canals.

The systems, methods and devices disclosed herein each have several innovative aspects, no single one of which is solely responsible for the desirable attributes disclosed herein. A variety of example systems and methods are provided below.

### Embodiment 1

A dental system to optically examine a dental structure, the system comprising:

a mechanical assembly sized and shaped to be inserted into a mouth of a patient;
an optical assembly configured to provide an illumination beam to a portion of the dental structure and to collect light from the portion of the dental structure, the optical assembly mounted on the mechanical assembly; and
an electronic processing system configured to:
analyze the information recovered from the collected light; and
determine a characteristic representative of the portion of the dental structure based on the analysis.

### Embodiment 2

The dental system of Embodiment 1, wherein the mechanical assembly is configured to provide a controllable path for moving the optical assembly in the mouth of the patient.

### Embodiment 3

The dental system of any of Embodiments 1-2, wherein the mechanical assembly comprises a track or a rail.

### Embodiment 4

The dental system of any of Embodiments 1-3, wherein the optical assembly comprises an optical fiber.

### Embodiment 5

The dental system of any of Embodiments 1-4, wherein the optical assembly can be moved with respect to the dental structure.

### Embodiment 6

The dental system of any of Embodiments 1-5, wherein the optical assembly is a part of a fluorescence spectroscopy system.

### Embodiment 7

The dental system of any of Embodiments 1-5, wherein the optical assembly is a part of a Raman spectroscopy system.

### Embodiment 8

The dental system of any of Embodiments 1-5, wherein the optical assembly is a part of an optical coherence tomography (OCT) system.

### Embodiment 9

The dental system of any of Embodiments 1-5, wherein the collected light is in a spectral region between a wavenumber of 420 cm⁻¹ and a wavenumber of 450 cm⁻¹.

### Embodiment 10

The dental system of any of Embodiments 1-5, wherein the collected light is in a spectral region between a wavenumber of 570 cm⁻¹ and a wavenumber of 610 cm⁻¹.

### Embodiment 11

The dental system of any of Embodiments 1-5, wherein the collected light is in a spectral region between a wavenumber of 940 cm⁻¹ and a wavenumber of 980 cm⁻¹.

### Embodiment 12

The dental system of any of Embodiments 1-5, wherein the collected light is in a spectral region between a wavenumber of 1020 cm⁻¹ and a wavenumber of 1065 cm⁻¹.

### Embodiment 13

The dental system of any of Embodiments 1-5, wherein the collected light is in a spectral region between a wavenumber of 2920 cm⁻¹ and a wavenumber of 2960 cm⁻¹.

### Embodiment 14

The dental system of any of Embodiments 1-5, wherein the information recovered from the collected light comprises a ratio of peak intensities in a first spectral region between a wavenumber of 420 cm⁻¹ and a wavenumber of 450 cm⁻¹ and a second spectral region between a wavenumber of 2920 cm⁻¹ and a wavenumber of 2960 cm⁻¹.

### Embodiment 15

The dental system of any of Embodiments 1-5, wherein the information recovered from the collected light comprises a ratio of peak intensities in a first spectral region between a wavenumber of 940 cm⁻¹ and a wavenumber of 980 cm⁻¹ and a second spectral region between a wavenumber of 2920 cm⁻¹ and a wavenumber of 2960 cm⁻¹.

### Embodiment 16

The dental system of any of Embodiments 1-15, wherein the electronic processing system is configured to generate a heat map of the portion of the dental structure based on the determined characteristic.

### Embodiment 17

A dental system to optically examine a dental structure, the system comprising:

a mechanical assembly sized and shaped to be inserted into a mouth of a patient;
an optical assembly configured to provide an illumination beam to a portion of the dental structure and to collect light from the portion of the dental structure, the optical assembly mounted on the mechanical assembly, the mechanical assembly configured to move the optical assembly relative to the dental structure to obtain a plurality of optical signals representative of a condition of the dental structure.

### Embodiment 18

The dental system of Embodiment 17, wherein the mechanical assembly comprises a track or a rail configured to facilitate movement of the optical assembly in the mouth of the patient.

### Embodiment 19

The dental system of Embodiment 17, wherein the optical assembly is laterally moved along an outer surface of the teeth to scan a plurality of locations on the outer surface of the teeth to detect caries.

### Embodiment 20

The dental system of Embodiment 17, wherein the optical assembly comprises an optical fiber configured to be inserted into a root canal or a periodontal pocket.

### Embodiment 21

The dental system of Embodiment 20, wherein the optical fiber is moved along a longitudinal axis of the root canal to scan a plurality of locations along a length of the root canal.

### Embodiment 22

The dental system of any of Embodiments 20-21, wherein the optical fiber is rotated about a longitudinal axis of the root canal to scan a plurality of locations along the inner surface of the root canal.

### Embodiment 23

The dental system of any of Embodiments 20-22, wherein light emitted from the optical fiber is directed along a direction transverse to the longitudinal axis of the root canal.

### Embodiment 24

The dental system of any of Embodiments 20-22, wherein light emitted from the optical fiber is directed along a direction parallel to the longitudinal axis of the root canal.

### Embodiment 25

The dental system of any of Embodiments 20-24, wherein mechanical assembly is integrated in a mouthguard sized and shaped to be inserted into the mouth of the patient.

### Embodiment 26

The dental system of any of Embodiments 20-25, further comprising an electronic processing system configured to determine a characteristic representative of the condition of the dental structure and generate a heat map of the dental structure based on the determined characteristic.

### Embodiment 27

The dental system of any of Embodiments 20-26, further comprising a display device, wherein the electronic processing system is configured to display the heat map on the display device.

### Embodiment 28

A dental system to optically examine a dental structure, the system comprising:

an optical assembly configured to provide an illumination beam to a portion of the dental structure and to collect light from the portion of the dental structure; and an electronic processing system configured to:

analyze the information recovered from the collected light;

determine a characteristic representative of the portion of the dental structure based on the analysis; and

render the determined characteristic on a display device as a heat map, the heat map displaying different values of the determined characteristic with different indicia on the display device.

### Embodiment 29

The dental system of Embodiment 28, wherein the optical assembly comprises an optical fiber.

### Embodiment 30

The dental system of any of Embodiments 28-29, wherein the optical assembly comprises a beam-steering system configured to scan the illumination beam across the portion of the dental structure.

### Embodiment 31

The dental system of any of Embodiments 28-30, further comprising a movement assembly configured to move the optical assembly from a first position in a mouth of a patient to a second position in the mouth of the patient.

### Embodiment 32

The dental system of Embodiment 31, wherein the movement assembly comprises a track or a rail placed in the mouth of the patient.

### Embodiment 33

A dental system to optically examine a dental structure, the system comprising:

an optical source configured to emit light;
an optical system configured to condition the light emitted from the optical source to generate an illumination beam;
an optical delivery system configured to deliver the generated illumination beam to a portion of a dental structure;
an optical collection system configured to collect light from the portion of the dental structure illuminated by the generated illumination beam;
an optical receiving system configured to receive the collected light, the optical receiving system configured to recover information from the collected light; and
an electronic processing system configured to:

analyze the information recovered from the collected light; and

determine a characteristic representative of the portion of the dental structure based on the analysis.

### Embodiment 34

The system of Embodiment 33, wherein the dental structure comprises a portion of a root canal of a tooth.

### Embodiment 35

The system of Embodiment 34, wherein the optical collection system comprises an optical fiber.

### Embodiment 36

The system of Embodiment 35, wherein the dental system is configured to cause the optical fiber to translate along a length of the root canal.

### Embodiment 37

The system of Embodiment 35, wherein the dental system is configured to cause the optical fiber to rotate about an axis of the root canal.

### Embodiment 38

The system of any of Embodiments 34-37, wherein the collected light comprises a fluorescence signal from the root canal of the tooth.

### Embodiment 39

The system of Embodiment 38, wherein the information recovered from the collected light comprises an intensity of the fluorescence signal from the root canal of the tooth at a plurality of wavelengths.

### Embodiment 40

The system of any of Embodiments 34-39, wherein the characteristic representative of the portion of the dental structure comprises an amount of bacteria in a portion of the dental structure or a cleanliness of a portion of the dental structure.

### Embodiment 41

The system of any of Embodiments 34-39, wherein the characteristic representative of the portion of the dental structure comprises an identification of a bacteria in a portion of the dental structure.

### Embodiment 42

The system of any of Embodiments 34-41, wherein the collection system is configured to collect light from a plurality of locations in the root canal of the tooth.

### Embodiment 43

The system of Embodiment 33, wherein the dental structure comprises a root canal of a tooth, a periodontal pocket or an enamel of the tooth.

### Embodiment 44

The system of Embodiment 43, wherein the optical system comprises an optical splitter configured to split the light emitted from the optical source along a reference arm and a signal arm.

### Embodiment 45

The system of Embodiment 44, wherein the collected light is configured to optically interfere with the light in the reference arm to generate an optical coherence tomography (OCT) image.

### Embodiment 46

The system of any of Embodiments 43-45, wherein the dental structure comprises the enamel of the tooth and the characteristic representative of the portion of the dental structure comprises caries, cavities, or cracks in the enamel of the tooth.

### Embodiment 47

The system of any of Embodiments 43-45, wherein the dental structure comprises the root canal and the characteristic of the portion of the dental structure comprises a length of the root canal.

### Embodiment 48

The system of any of Embodiments 43-45, wherein the dental structure comprises a periodontal pocket and the characteristic of the portion of the dental structure comprises a morphology of the periodontal pocket.

### Embodiment 49

The system of Embodiment 33, wherein the dental structure comprises an enamel of the tooth.

### Embodiment 50

The system of Embodiment 49, wherein the collected light comprises Raman scattered light.

### Embodiment 51

The system of Embodiment 50, wherein the optical collection system comprises an optical filter having a passband that attenuates the illumination beam.

### Embodiment 52

The system of Embodiment 51, wherein the characteristic of the portion of the dental structure comprises demineralization of the enamel.

### Embodiment 53

The system of Embodiment 33, further comprising a display device, wherein the electronic processing system is configured to display the determined characteristic on the display device.

### Embodiment 54

The system of Embodiment 53, wherein the electronic processing system is configured to display the determined characteristic on the display device as a heat map, the heat map displaying different values of the determined characteristic with different indicia on the display device.

### Embodiment 55

The system of Embodiment 54, wherein the electronic processing system is configured to overlay the heat map over an image of the examined portion of the dental structure.

### Embodiment 56

The system of Embodiment 33, further comprising a mounting assembly and a housing configured to be attached to the mounting assembly, wherein the housing comprises at least one of the light delivery system or the light collection system.

### Embodiment 57

The dental system of any one of Embodiments 33 to 56, further comprising a treatment system configured to clean the dental structure.

### Embodiment 58

The dental system of Embodiment 57, wherein the treatment system comprises a pressure wave generator configured to generate pressure waves in a treatment fluid having energy sufficient to clean the dental structure.

### Embodiment 59

The dental system of Embodiment 58, wherein the pressure wave generator comprises a liquid jet device.

### Embodiment 60

The dental system of any one of Embodiments 58-59, wherein the treatment system comprises a fluid platform configured to position a distal end of the pressure wave generator within a chamber of a tooth, the pressure wave generator configured to clean a root canal of the tooth.

### Embodiment 61

The dental system of any one of Embodiments 58-59, wherein the treatment system comprises a fluid motion generator configured to generate a swirling flow profile of fluid to clean the dental structure.

### Embodiment 52

The dental system of any one of Embodiments 58-59, wherein the treatment system comprises a chamber configured to be positioned against a tooth over a carious region on an exterior surface of the tooth, the pressure wave generator configured to clean the carious region.

### Embodiment 63

The dental system of any one of Embodiments 57-62, further comprising a console, the treatment system and the electronic processing system disposed in or on the console.

### Embodiment 64

The dental system of any of Embodiments 33-63, wherein the collected light is in a spectral region between a wavenumber of 420 cm⁻¹ and a wavenumber of 450 cm⁻¹.

### Embodiment 65

The dental system of any of Embodiments 33-63, wherein the collected light is in a spectral region between a wavenumber of 570 cm⁻¹ and a wavenumber of 610 cm⁻¹.

### Embodiment 66

The dental system of any of Embodiments 33-63, wherein the collected light is in a spectral region between a wavenumber of 940 cm⁻¹ and a wavenumber of 980 cm⁻¹.

### Embodiment 67

The dental system of any of Embodiments 33-63, wherein the collected light is in a spectral region between a wavenumber of 1020 cm⁻¹ and a wavenumber of 1065 cm⁻¹.

### Embodiment 68

The dental system of any of Embodiments 33-63, wherein the collected light is in a spectral region between a wavenumber of 2920 cm⁻¹ and a wavenumber of 2960 cm⁻¹.

### Embodiment 69

A method of determining a characteristic of a portion of a dental structure based on an optical examination of the portion of the dental structure, the method comprising:

directing an illumination beam to a portion of a dental structure from an optical source included in an optical system;
receiving light from the portion of the dental structure at an optical receiver included in the optical system;
analyzing the received light using an electronic processing system in electrical communication with the optical system;
determining a characteristic representative of the portion of the dental structure, using the electronic processing system; and
providing an output based on the determined characteristic on an output device.

### Embodiment 70

The method of Embodiment 69, wherein the optical system comprises a fluorescence spectroscopy measurement system.

### Embodiment 71

The method of Embodiment 70, wherein the portion of the dental structure comprises a root canal.

### Embodiment 72

The method of Embodiment 71, wherein the determined characteristic comprises at least one of an amount of bacteria in the root canal, an identification of bacteria in the root canal, or a metric associated with cleanliness of the root canal.

### Embodiment 73

The method of Embodiment 69, wherein the optical system is a Raman spectroscopy measurement system.

### Embodiment 74

The method of Embodiment 73, wherein the portion of the dental structure comprises an enamel.

### Embodiment 75

The method of Embodiment 74, wherein the determined characteristic comprises a demineralization index.

### Embodiment 76

The method of Embodiment 69, wherein the optical system is an optical coherence tomography system.

### Embodiment 77

The method of Embodiment 76, wherein the portion of the dental structure comprises an enamel, a root canal or a periodontal pocket.

### Embodiment 78

The method of Embodiment 77, wherein the determined characteristic comprises a length of the root canal.

### Embodiment 79

The method of Embodiment 77, wherein the determined characteristic comprises caries, cavities or cracks in the enamel.

### Embodiment 80

The method of Embodiment 77, wherein the determined characteristic comprises a morphology of the enamel, the root canal or the periodontal pocket.

### Embodiment 81

The method of Embodiment 69, wherein the output comprises the determined characteristic, and the output device comprises a display device.

### Embodiment 82

The method of Embodiment 71, further comprising displaying the determined characteristic on the display device as a heat map.

### Embodiment 83

The method of Embodiment 82, further comprising overlaying the heat map over an image of the examined portion of the dental structure.

### Embodiment 84

The method of any of Embodiments 69-83, wherein directing an illumination beam to a portion of a dental structure comprises scanning the illumination beam across the portion of the dental structure.

### BRIEF DESCRIPTION OF THE DRAWINGS

The following drawings and the associated descriptions are provided to illustrate embodiments of the present disclosure and do not limit the scope of the claims.

FIG. 1 schematically illustrates an embodiment of an optical system configured to excited fluorescence in the protoporphyrin and/or porphyrin molecules.

FIG. 2 shows spectra collected from different internal and external locations on a freshly extracted diseased tooth.

FIG. 3 shows the fluorescence spectra for F. nucleatum bacteria when illuminated by an excitation light.

FIG. 4 shows the fluorescence spectra for P. intermedia bacteria when illuminated by an excitation light.

FIG. 5 illustrates an optical system configured to perform a Raman spectroscopic examination of a tooth.

FIG. 6 illustrates an embodiment of an optical coherence tomography (OCT) system configured to perform an optical examination of a tooth.

FIG. 7 schematically illustrates an embodiment of an OCT system configured to obtain cross-sectional images (in the radial-axial plane) of the surfaces of a root canal.

FIG. 8 schematically illustrates an embodiment of an OCT system that can be used to visualize the periodontal space.

FIG. 9 schematically illustrates an example heat map showing the bacterial load in various portions of a root canal.

FIGS. 10A-10C depict an example of a mounting assembly.

FIG. 11 is a schematic system diagram of a dental system.

FIG. 12 schematically illustrates an example of a treatment system for treating (e.g., cleaning) a tooth with a pressure wave generator.

FIG. 13 and FIG. 14 are graphs that schematically illustrate possible examples of acoustic power that could be generated by different embodiments of the pressure wave generators disclosed herein.

FIG. 15 schematically illustrates an example of a treatment system for treating (e.g., cleaning) a tooth with a fluid motion generator that comprises a pressure wave generator, according to various embodiments.

FIG. 16 schematically illustrates an example of a treatment system for treating (e.g., cleaning) a carious region on an exterior surface of a tooth.

### DETAILED DESCRIPTION

Although certain preferred embodiments and examples are disclosed herein, inventive subject matter extends beyond the specifically disclosed embodiments to other alternative embodiments and/or uses of the inventions, and to modifications and equivalents thereof. Thus, the scope of the inventions herein disclosed is not limited by any of the particular embodiments described below. For example, in any method or process disclosed herein, the acts or operations of the method or process may be performed in any suitable sequence and are not necessarily limited to any particular disclosed sequence.

For purposes of contrasting various embodiments with the prior art, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Various embodiments described in this application include optical systems and methods to examine a portion of a dental tissue. As used herein, dental tissue can include the different components or layers of a tooth including but not limited to enamel, dentin, cementum and pulp. As used herein, dental tissue can also include gingivae or gums. The optical systems and methods described herein can detect caries and tooth defects (e.g., cracks), estimate the amount of bacteria in the root canal of one or more teeth of the patient, measure the working length of a root canal, characterize the structure and architecture of inner canal wall structure of the one or more teeth, visualize and diagnose peridontium pathologies and/or visualize apical region of a root canal. The systems and methods described herein can have several advantages. For example, the optical system and methods described herein can provide a non-destructive and a non-invasive method for examining dental tissue. As another example, the systems and methods described herein can examine the one or more teeth quickly as compared to other methods. For example, optical measurement of one or more teeth of a patient can be obtained in 10 seconds or less using the optical system and methods described herein. The optical systems and methods described herein can be configured to be compact by using miniaturized optical components and/or optical fibers.

### Optical Interrogation of a Root Canal

Root canal bacteria can endogenously bio-synthesize protoporphyrin and/or porphyrin molecules after consuming root canal pulpal tissue. The protoporphyrin and/or porphyrin molecules are located wherever bacteria are present inside a root canal. The optical systems and methods described herein can be used to optically detect the amount of protoporphyrin and/or porphyrin molecules and/or the bacteria present in the root canal. For example, the protoporphyrin and/or porphyrin molecules can be detected via spectroscopic methods. Without any loss of generality, the cleanliness of a root canal depends on the amount of bacteria (or bacterial load) present in the root canal. A root canal with a lower bacterial load can be considered to be cleaner than a root canal with a higher bacterial load. Accordingly, the root canal of a patient's teeth can be optically interrogated to determine the cleanliness of a root canal.

Systems and methods employed to optically interrogate a root canal can have several advantages. For example, the entire root canal all the way down to the apex can be interrogated using optical techniques. As another example, optical techniques of interrogating a root canal can provide a non-destructive and a non-invasive method for examining the root canal. Additionally, optical examination of a root canal can done quickly. For example, measurements that are related to cleanliness of the root canal can be obtained in less than 10 second (e.g., as little as 1-2 seconds). Optical fiber based systems and methods of examining root canals can be compact since optical fibers can have a small diameter (e.g., diameters between approximately 100 microns and approximately 300 microns). Thus, optical fiber based systems and methods of examining root canals can work with non-instrumented canals. As used herein, a non-instrumented canal refers to a root canal that is prepared without removal of endogenous tissue (e.g. dentin). Accordingly, a tooth with non-instrumented canal can have higher structural integrity as compared to an instrumented canal which is prepared by removal of endogenous tissue (e.g. dentin) by filing or some other method of removing tissue. Additionally, the systems and methods of optically interrogating the root canal discussed herein can provide quantitative measurements that can be used to classify the cleanliness of the root canal.

One method of determining the bacterial load in the root canal comprises fluorescence spectroscopy. The protoporphyrin and/or porphyrin molecules synthesized by the bacteria in the root canal can fluoresce when illuminated by light having wavelengths (e.g., wavelengths in the range between about 280 nm and about 650 nm). Without any loss of generality, the fluorescence process involves elevation of the protoporphyrin and/or porphyrin molecules to a higher energy state as a result of absorption of the light at the excitation wavelength followed by a spontaneous decay to a distribution of rotational and vibrational energy states within the same or lower energy state. When decaying from the higher energy level to the lower energy level, the difference in the energy between the two energy levels is released in the form of broadband emission of light in the visible or near infrared spectrum.

The presence and/or amount of bacteria present in the root canal can also be detected via Raman spectroscopy which is described in greater detail below. Without any loss of generality, Raman spectroscopy is an inelastic scattering phenomena and is capable of providing biochemical and morphological information. Scattering is predominantly an elastic phenomena, whereby the scattered light has the same frequency as the incident light; however, a portion of the scattered light can be attributed to inelastic scattering which has a different frequency from the incident light. By detecting and analyzing the scattered light at a different frequency from the incident light, information about the scattering material can be obtained.

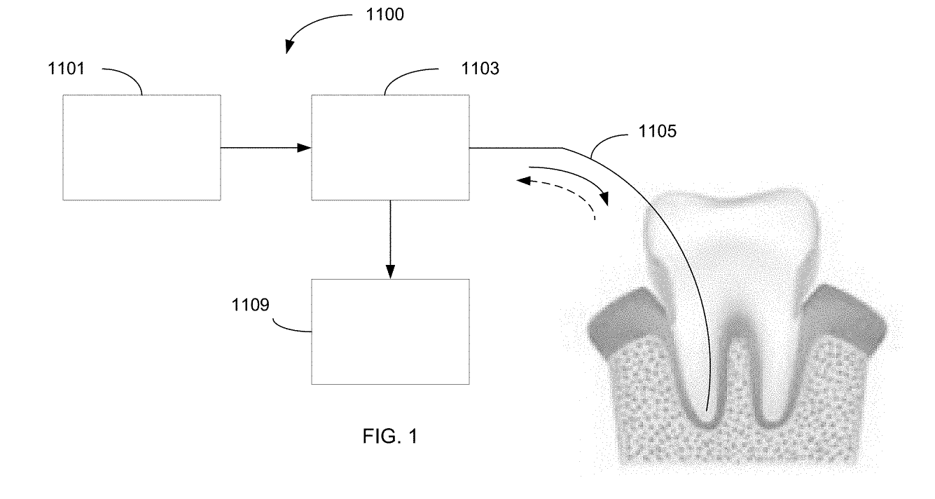

One embodiment of an optical system 1100 configured to excited fluorescence in the protoporphyrin and/or porphyrin molecules is depicted in FIG. 1. The optical system 1100 can include a combination of fiber optic and freespace optical elements. The optical system 1100 comprises an optical source 1101. The optical source 1101 can comprise a laser, one or more laser diodes and/or one or more light emitting diodes (LEDs). The optical source 1101 can be operated in a continuous (CW) mode or a pulsed mode. In some embodiments, the optical source 1101 can comprise a monochromatic source that outputs light having a single wavelength. In some embodiments, the optical source 1101 can comprise a broadband source that outputs broadband light in a spectral range. In some embodiments, the optical source 1101 can comprise a tunable laser whose output wavelength can be controlled. The optical source 1101 can be configured to output light having a wavelength in one or more of the ultraviolet spectral range, the visible spectral range and/or the near-infrared spectral range. For example, the optical source 1101 can be configured to output light having a wavelength between about 250 nm and about 430 nm, between about 400 nm and about 510 nm, between about 500 nm and about 550 nm, between about 520 nm and about 610 nm, between about 580 nm and about 650 nm, between about 630 nm and about 780 nm, between about 650 nm and about 980 nm or any range/sub-range defined by any of these wavelength ranges.

The light output from the optical source 1101 can be conditioned by an optical system 1103 comprising a plurality of optical components. For example, the optical system 1103 can comprise a short-pass dichroic filter, configured to transmit the laser light and reflect the longer fluorescence wavelengths. As another example, the optical system 1103 can comprise one or more collimating and/or focusing lenses configured to focus the light from the optical source 1101 onto a portion of a tooth. As yet another example, the optical system 1103 can comprise a dynamic optical component, such as, for example, a galvanometer or a micro-electro mechanical system (MEMS) based device that can be controlled to focus light from the optical source 1101 on different locations of the tooth. In various embodiments of the optical system 1100 configured to examine the root canal, the light from the optical source 1101 can be coupled into an optical fiber 1105 using one or more lenses (e.g., a focusing lens). The spectrum of the light from the optical source 1101 can be tailored using a band-pass filter that transmits the light output from the optical source 1101 and reduces or eliminates any tails in the spectrum of the light output from the optical source 1101 prior to being coupled into the optical fiber 1105. The optical fiber 1105 can be configured as a flexible cable, which is capable of being inserted into the root canal and is capable of being navigated along the length and the curvature of the root canal. To estimate the cleanliness of the root canal, the optical fiber 1105 can be positioned at one or more locations within the root canal. At each or some of the one or more location, the user (e.g., dentist/dentist's assistant) can activate the optical source 1101, for example, by pressing a button or some other way to excite fluorescence. In some embodiments, the optical fiber 1105 can be configured to output light along a direction parallel to the optical axis of the optical fiber 1105 (or the axis of the root canal) which can extend along the length of the optical fiber 1105. In such embodiments, the light output from the optical fiber 1105 is emitted from the end face of the optical fiber that is inserted into the root canal along a direction normal to the end face. In some embodiments, the light output from the optical fiber 1105 can be directed in a radial direction (e.g., normal or at an angle with respect to the optical axis of the optical fiber 1105 or the axis of the root canal). This configuration can be referred to as “side-firing.” One advantage of the side-firing configuration is the ability to improve optical sampling on the root canal walls, where bacteria typically reside in the form of biofilm on the dentinal walls and inside dentinal tubules. In an example embodiment comprising the side-firing configuration includes an optical fiber having a 400 micron core diameter that achieves side firing via total internal reflection on the distal end (the end closer to the root canal). In some embodiments, the light emitted from the optical fiber 1105 can be directed laterally with respect to the axis of the canal. Lateral direction of light can be achieved by providing a lens having a beveled surface at the distal end of the optical fiber 1105 (the end inserted into the root canal) or by providing a reflection surface orientated at an angle with respect to the axis of the optical fiber 1105 (or axis of the root canal). This configuration can be similar or identical to the side-firing configuration. An advantage of directing the light along a direction lateral to the axis of the canal is that the lateral features of the tooth structure, such as dentin tubules or lateral canals, can be interrogated for bacterial presence.

Any fluorescence, excited by the activation of the optical source 1101, is collected by the optical fiber 1105 and directed towards the optical system 1103. The optical fiber 1105 can be configured to have a high numerical aperture to efficiently collect as much of the fluorescence signal as possible. For example, the distal end of the optical fiber 1105 (e.g., the end inserted into the root canal) can comprise a lens configured to enhance the collection efficiency. The lens can be a ball lens or a half-ball lens. The lens can have high refractive index. Additionally, the lens can comprise a high impact resistance material such as ruby or sapphire. The collected fluorescence signal can be directed by a dichroic filter in the optical system 1103 along a receive path towards an optical receiver 1109. The dichroic filter in the optical system 1103 can be configured to transmit light from the optical source 1101 and reflect the fluorescence signal. In order to collect fluorescent signal corresponding to a particular wavelength region, with a demonstrable ability to provide an indication of bacterial presence, the collection optical train can comprise a specialized multi-bandpass filter disposed in the receive path. The optical receiver 1109 can comprise a photodetector and/or a spectrometer. In various embodiments, the fluorescent signal can be collected using other types of photodetectors such as photomultiplier tubes (PMTs) or photodiodes. In various embodiments, the optical receiver 1109 can provide a digital signal of fluorescent intensity as a function of wavelength.

The optical system 1100 can be operated in “integrated” or “single shot” mode. In this mode, the optical source 1101 can be pulsed optical source that emits optical pulses at fixed or regular time intervals. Fluorescence signal can be collected from many different locations while the fiber optic probe is positioned at different portions inside the root canal. In some embodiments, different pulses can have different spectral contents. The fluorescence spectra obtained for a plurality of the pulses can be averaged to provide an integrated measurement of the entire tooth's cleanliness. In some embodiments, the user (e.g., the dentist/dentist's assistant) can operate the device such that a reading is provided each time the laser is activated, thereby providing the user with spatial discretization of cleanliness within the tooth.

Some embodiments of the optical system 1100 can be entirely (or substantially entirely) fiber-optic based design with the filters miniaturized and placed inside fiber-optic housing. Various embodiments of the optical system 1100 can comprise an optical source 1101 that can emit a plurality of wavelengths to induce fluorescence from multiple bacterial species. The excitation and collection sub-systems can be synchronized to sequentially excite and collect fluorescence from different bacterial strains.

The optical receiver 1109 can comprise an electronic processing system configured to analyze the obtained fluorescent spectra. The electronic processing system can be further configured to estimate the bacterial load in various portions of the root canal and/or provide a metric associated with the cleanliness of the root canal. The electronic processing system can be configured to increase the signal-to-noise ratio of the obtained fluorescence spectra by using various signal processing techniques. For example, the signal-to-noise ratio of the obtained fluorescence spectra can be increased by using digital filtering algorithms such as a Savitsky-Golay filter and/or smoothing of the spectra using simple methods such a window average. Improving the signal-to-noise ratio of the obtained fluorescence spectra can increase the reliability of detecting the presence of bacteria in the root canal and estimating the cleanliness of the root canal.

Spectral analysis can be performed by signal processing algorithms to remove background signal due to autofluorescence from endogenous species (dentin, enamel, etc) or species present post-root canal treatment (RCT), such as, for example, EDTA, water, sodium hypochloride, etc. The result of this analysis can be a “background corrected” spectrum, representative of the photonic signal due only to the porphyrin fluorescence.

In various embodiments, it may be desirable to reduce the data size via software binning which can potentially reduce hardware-related costs, reduce data rates and/or reduce computational processing time associated with the optical detection methods disclosed herein. As fluorescence spectra can be broadband, spectral binning can be implemented with reduced loss in data accuracy.

### Bacteria Identification

Using linear algebraic mathematical operations, a measured spectra, representing the linear superposition of multiple fluorophores, can be decomposed into individual spectral profiles corresponding to one of the multiple fluorophores to determine the presence of different bacteria. Spectral decomposition can yield relative semi-quantitative concentrations of bacterial populations; in-vitro calibration of fluorescent intensity as a function of bacterial concentration can be used for true quantification. Various spectral decomposition methods are discussed below.

The fluorescence spectra obtained by the optical system 1100 can be a combination of all possible interrogated species that generate fluorescence at wavelengths of the excitation light. These could be many different strains of bacteria, enamel, dentin, restorative material, obturation material. The following method describes a way to deconstruct the measured, integrated spectra into its “basis” or “constituent” components in a technique known as spectral decomposition.

The system can be modeled by equation (1)

M=SeC^{T}+ε (1)

Where M=measured spectrum with dimension equal to the number, n, of wavelength datapoints, which depends on the number of x-axis pixels and the x-bin setting, n can be given by equation (2) below:

n=1360/x_{bin} (2)

In equation (1), S is the basis spectra matrix with dimension n×d, where d is the number of species, c is the concentration vector with dimension d×1, and e is the noise. The measured spectrum can also be corrected by subtracting any background signal that was present.

The Gauss-Markov theorem states that a linear regression model: y=Xb+ε, where ε is the noise then f=(X^{T}X)⁻¹X^{T} is the function of X and b that minimizes the sum of the squared residuals.

Initially if the noise is ignored:

(S^{T}S)⁻¹s^{T}m=(S^{T}S)⁻¹(S^{T}S)c

(S^{T}S)⁻¹(S^{T}S)=I

∴c=(S^{T}S)⁻¹S^{T}m

The quantity K given by equation (3) is the pseudo-inverse of Se_{bin}, and can be pre-computed.

K=(S^{T}S)⁻¹S^{T} (3)

Thus, quantification may be performed in realtime by using equation (4)

Ĉ^{T}=KM_{bin} (4)

The above described spectral decomposition technique can be applied even if M_{bin} contains multiple spectra, for example, a plurality of columns with each column representing a spectrum.

### Experimental Results

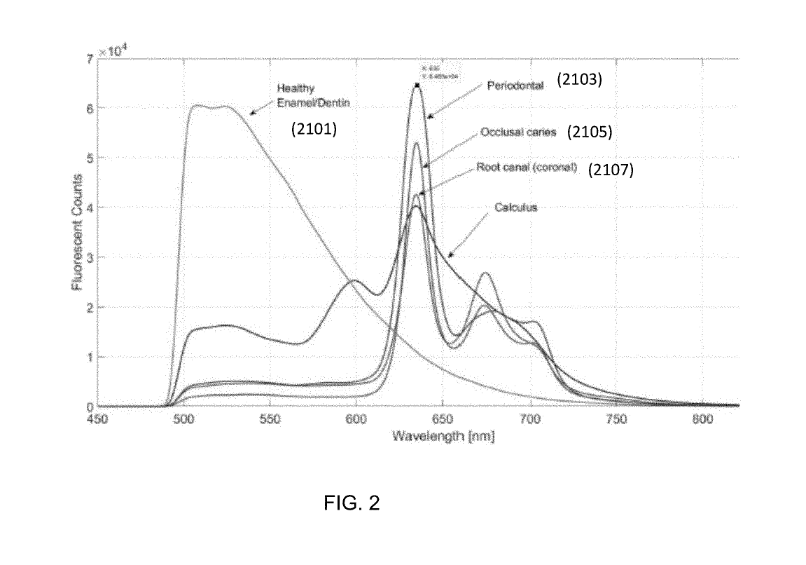

FIG. 2 shows spectra collected from different internal and external locations on a freshly extracted diseased tooth. The healthy hard tissue spectrum (curve 2101) is a broadband, monotonically decaying signal, which contrasts with spectra collected from locations with pathology with well-defined, prominent peaks superimposed onto a broadband signature. For example, curve 2103 corresponds to the spectrum obtained from the periodontal region, curve 2105 corresponds to the spectrum obtained from occlusal caries, curve 2107 corresponds to the spectrum obtained from root canal. These peaks and the associated fluorescence profiles are from porphyrins, which are organic compounds occurring as digestive products of endodontic bacteria such as Enterococcus Faecalis and Prevotella Intermedia.

The differences in spectral shape provide encouraging visual evidence of the diagnostic capabilities of the fluorescence spectroscopic method of estimating bacterial load in the root canal. An overview of a diagnostic protocol used to estimate the cleanliness of the root canal is described below:

1. Basis spectra can be measured for known species that fluorescence at the excitation laser wavelength of choice. Examples of these species include Protoporphyrin IX, Coproporphyrin III, healthy enamel and healthy dentin.

2. Based on characterization studies previously performed, identify the limits of detection for each basis species in the presence of other species, corresponding to the minimal signal that can be detected to determine the presence of a particular species.

3. A spectrum, or series of spectra, are collected from locations inside or outside a tooth. These spectra are then processed using the mathematical linear algebra analysis described above to assign contributions from the basis components using the limit of detection values.

4. The assigned contributions are used to provide output to the user designating if the measured location is “clean” or “dirty”, and the level of infection quantified into qualitative categories such as “high”, “medium” or “low”.

### In Vitro Experiments

Many bacterial strains can be present in vital root canal pulp tissue and theoretically any of these bacterial strains can present pathological concern to cause pulp infection and possible periapical infection. However, in practice only a few bacterial species are present in infected root canals. Previous studies have demonstrated that the root canal microbiology is typically dominated by anaerobic bacteria, corresponding to a commensurate reduction in facultative species. In terms of fluorescence detection, the species found in infected root canals can be simplistically described by two main categories: those that emit fluorescence in the green portion of the visible spectrum and those that emit fluorescence in the red.

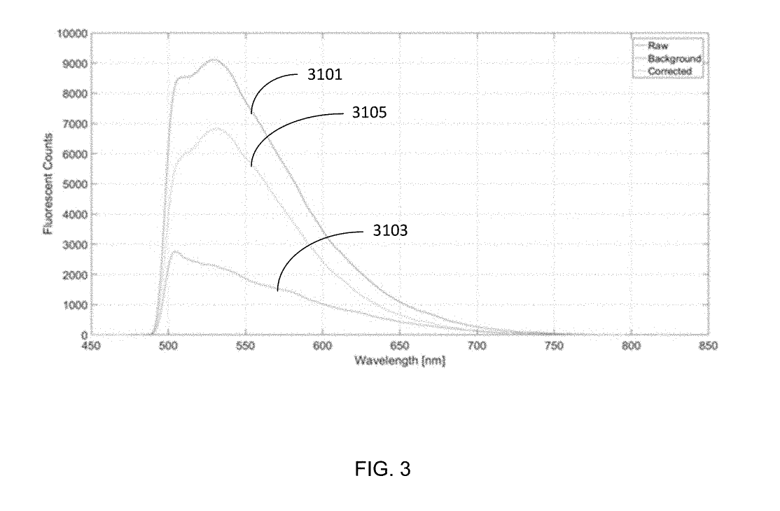

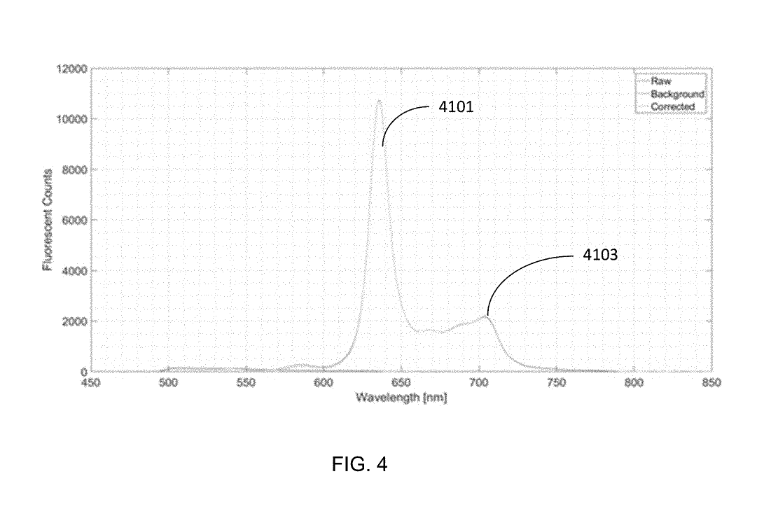

The results of an in vitro study conducted to demonstrate the ability to detect both types of fluorescence using the systems and methods discussed above are presented. Two species, Fusobacterium nucleatum and Prevotella Intermedia, were cultivated on agar plates and, after an incubation period, placed into a water solution for measurement with the fiber-optic probe. These species are the two most frequently isolated in root canals, found in 48% and 34% of root canals respectively. The spectra were collected with the prototype by immersing the probe in vials containing the solution and acquiring data at clinically relevant exposure times (100 ms). Spectra were also obtained of the solvent alone and served as background. The background spectra was then subtracted from the raw spectra, yielding a corrected spectra. FIG. 3 shows the results for F. nucleatum and FIG. 4 shows the results for P. intermedia. In FIG. 3, curve 3101 represents the raw fluorescent spectrum, curve 3103 represents the background fluorescence and curve 3105 represents the corrected fluorescent spectrum obtained by subtracting the background fluorescence from the raw spectrum. In FIG. 4, curve 4101 represents the corrected fluorescent spectrum obtained by subtracting the background fluorescence from the raw spectrum.

In the case of P. intermedia, the red fluorescence peak is clearly observed in the curve 3105. In the case of the “green” species, F. nucleatum, a less intense, broader “hump-like” structure 4103 can be observed in curve 4101. The shape of the spectra, with a small peak superimposed onto a spectrally broad signal, may suggest that other sources of fluorescence are present such as auto-fluorescence generated by the fiber-optic glass. This auto-fluorescence can be eliminated via material selection in more refined prototype version.

### SUMMARY

The optical system described above utilizes fluorescence spectroscopic methods to optically interrogate a root canal and determine the cleanliness of a root canal. In other embodiments, the fluorescence spectroscopic methods can optically interrogate other treatment regions (such as caries on an outer surface of the tooth, gingival pockets, etc.) to examine the cleanliness of the treatment region. Additionally, the fluorescence spectroscopic methods can be used to estimate the bacterial load in different portions of the root canal and/or identify the various bacteria present in the root canal. The optical system configured to determine the cleanliness of the root canal can be integrated with a device that is configured to clean root canals to determine the efficacy of the cleaning process. The system to obtain fluorescence spectra from different portions of the root canal can comprise a tunable laser configured to be operated in a CW mode or a pulsed mode. For example, the tunable laser can be configured emit a first pulse train having a first wavelength at a first time interval and a second pulse train having a second wavelength at a second time interval. The first pulse train can be configured to excite protopophyrins synthesized by a first bacteria and the second pulse train can be configured to excite protopophyrins synthesized by a second bacteria. Fluorescence from the protopophyrins synthesized by the first bacteria can be collected in the first time interval and fluorescence from the protopophyrins synthesized by the first bacteria can be collected in the second time interval. Thus, the fluorescence data collection from different wavelengths can be synchronized to the duration of the pulses for the different wavelengths. In some embodiments, the optical source can be operated in the continuous mode. Fluorescence data can be collected from different heights along the length of the root canal to determine the cleanliness along the length of the root canal.

### Raman Spectroscopy

Various optical systems and methods to examine a tooth contemplated by this application to comprises an optical system configured to perform a Raman spectroscopy (RS) on the tooth. An optical system configured to perform Raman spectroscopy can advantageously detect early carious lesions. For example, demineralization of the hydroxyapatite matrix, which is initiated by acids produced by oral bacterial, alters chemical bonds which can be detected using the Raman spectroscopic technique. Demineralization can eventually lead to clinical presentation associated with caries such as white spot lesions and cavitation (holes) as the acid continues to deteriorate the matrix. Without subscribing to any particular theory, Raman spectroscopy can measure the phenomena of inelastic scattering, in which scattered light undergoes an energy shift relative to the incident light. The energy shift that results from inelastic scattering can translate to a change in wavelength, which can be detected and measured. Without subscribing to any particular theory, the energy shift can occur as a result of excitation of molecules in various portions of the tooth being examined to higher vibrational and/or rotational energy levels.

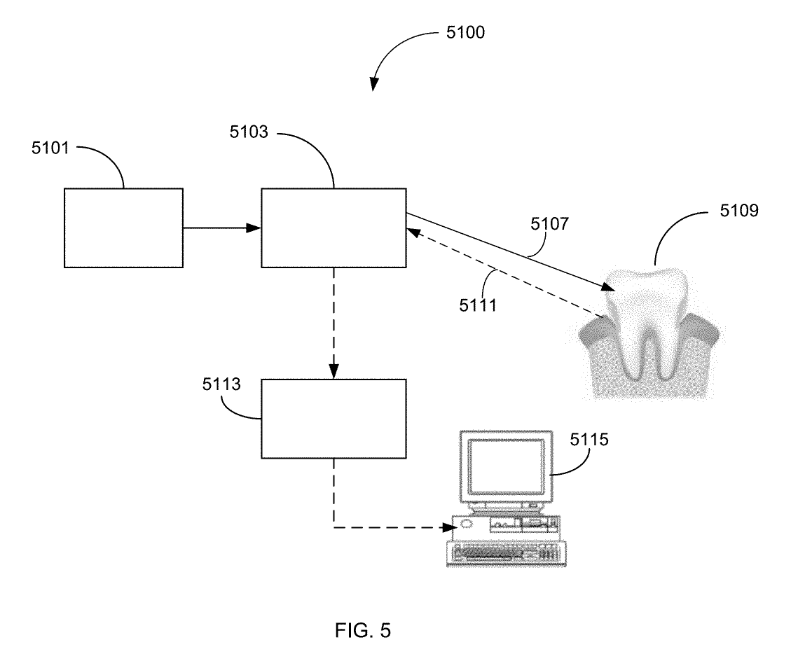

FIG. 5 illustrates an optical system 5100 configured to perform a Raman spectroscopic examination of a tooth. The system 5100 comprises an optical source 5101 configured to emit an optical beam comprising light at one or more wavelengths, conditioning optics 5103 configured to condition the optical beam and output a conditioned illumination beam 5107 to at least a portion of a tooth 5109. In various implementations, the conditioned optical beam 5107 can be directed to an entire tooth or a portion of the tooth (e.g., enamel, a portion at or below the gumline surrounding the tooth, a periodontal pocket, or any other portion of the tooth). In the illustrated embodiment, the conditioned optical beam 5107 is directed at an exterior surface of the tooth 5109 to detect or monitor a progression of caries on an external surface of the tooth 5109. In other embodiments, the conditioned optical beam 5107 can be directed at inner surfaces of the tooth 5109 (e.g., the pulp chamber, root canal(s), etc.) after cleaning to determine the cleanliness of the tooth. The Raman scattered light 5111 from one or more portions of the tooth 5109 can be collected by the collection optics included in the conditioning optics 5103 and directed towards an optical receiver system 5113. The light received at the optical receiver system can be analyzed by an electronic processing system 5115 configured to determine one or more characteristics that is representative of a constituent of the teeth and/or a morphology of a constituent of the teeth.

The optical system 5100 illustrated in FIG. 5 can be used in facilities that provide dental care. The optical system 5100 can be configured to be mobile and easily portable. The system can be used and operated by dentists/dental hygienists to obtain Raman spectroscopic data of at least a portion of a tooth of a patient. In some embodiments, the optical system 5100 can be automated and designed in such a manner that it can be operated by an approximately untrained operator. In some embodiments, the optical system 5100 can be setup and operated in a relatively short period of time.

The optical source 5101 can comprise a near-infrared (NIR) source of radiation. For example, in various embodiments, the optical source 5101 can be configured to emit radiation in a wavelength range between approximately 700 nm and approximately 1.5 micron. For example, the optical source 5101 can be configured to emit radiation in a wavelength range between approximately 700 nm and approximately 850 nm, between approximately 800 nm and approximately 900 nm, between approximately 850 nm and approximately 980 nm, between approximately 900 nm and approximately 1.1 micron, between approximately 1.0 micron and approximately 1.3 micron, or any wavelength in a range/sub-range defined by any of these values. Various embodiments of the optical source 5101 can comprise a laser, a laser diode, a light emitting diode (LED) or any other source capable of emitting radiation in the NIR wavelength range. The optical source 5101 can be operated in continuous mode or in pulsed mode. In some embodiments, electric power to the optical source 5101 can be supplied from an electrical power supply line. In various embodiments, electrical power to the optical source 5101 can be supplied by a voltage regulator. In some embodiments, electrical power to the optical source 5101 can be supplied by a battery pack. In various embodiments, an electronic control system can be used to control the optical source 5101. The electronic control system can be configured to switch the optical source 5101 on or off, and/or control one or more parameters (e.g., average optical power, spot size, output wavelength, etc.) of the output optical beam 5107. In some embodiments, the electronic control system can be used to alternate between continuous and pulsed mode of operation. In various embodiments, the electronic processing system 5115 can comprise the electronic control system configured to control the optical source 5101.

As discussed above, the light emitted from the optical source 5101 can be conditioned by conditioning optics 5103. The conditioning optics 5103 can comprise one or more optical elements (e.g., lenses, optical beam splitters, dichroic mirrors, reflectors, polarization controllers, polarizers, retarders, prisms, wavelength filters, spatial filters, such as, for example, a pin-hole or an aperture, galvanometer, resonant or micro electromechanical (MEMs) mirror system, optical attenuators, or a combination of any of these components). The conditioning optics 5103 can be configured to tailor the light emitted from the optical source 5101 to produce a light beam having a desired wavelength and a desired spot size on at least a portion of the tooth 5109.

The light beam at the output of the conditioning optics 5103 is delivered to at least a portion of the tooth 5109. The light beam output from the conditioning optics 5103 can be delivered to at least the portion of the tooth in freespace. As discussed above, the conditioning optics 5103 can comprise a beam-steering system (e.g., a galvanometer or a MEMS based scanning system) that can be used to scan the light beam across the surface of a portion of the tooth 5109. In some embodiments, an optical probe can be connected to the output of the conditioning optics 5103 to deliver the light beam. The optical probe can be flexible or rigid. The optical probe can comprise a resilient material that can be bent into one or more desired shapes. In various implementations, the optical probe can comprise metal, plastic, silica, or polymer. In various implementations, the optical probe can comprise an optical fiber.

In various embodiments, the elements of the conditioning optics 5103 can be configured to produce a light beam 5107 having desired optical properties (e.g., a desired optical spot size, a desired wavelength or a desired range of wavelengths, a desired optical power, etc.) on the portion of the tooth 5109 to be examined. The light beam 5107 can optically interact with the portion of the tooth 5109 to be examined. For example, the light beam 5107 incident on the portion of the tooth 5109 being examined can be scattered non-elastically (e.g., by the phenomenon of Raman scattering) by the constituents of the portion of the tooth 5109 being examined as scattered beam 5111. The wavelength of the scattered beam 5111 can be different from the wavelength of the incident light beam 5107 due to the Raman frequency signal shift discussed above. The scattered beam 5111 can be collected by the conditioning optics 5103 and directed towards the optical receiver system 5113.

The conditioning optics 5103 can comprise an optical beam splitter (e.g., a dichroic mirror) that directs the scattered beam 5111 towards the optical receiver system 5113 along a receive path. The optical beam splitter (e.g., the dichroic mirror) can be configured to transmit the light from the optical source 5101. The optical receiver system 5113 can comprise one or more optical filters (e.g., optical bandpass filters) and one or more photodetectors. The one or more optical filters can be configured to transmit a portion of the collected light in one or more spectral regions corresponding to the spectral regions where the information from the optical spectrum can be correlated to the constituents of the teeth. For example, in some implementations, the one or more optical filters can be configured to transmit a portion of the collected light in one or more spectral regions where the information from the optical spectrum can be correlated with the mineralization level of enamel, such as the bending and/or stretching mineralization index. For example, the one or more optical filters can be configured to transmit light in a spectral region between a wavenumber of 430 cm⁻¹ and a wavenumber of 2941 cm⁻¹ corresponding to the mineralization level of enamel. As another example, the one or more optical filters can be configured to transmit light in a spectral region between a wavenumber of 960 cm⁻¹ and a wavenumber of 2941 cm⁻¹ corresponding to bending and/or stretching mineralization index. In various embodiments, the one or more optical filters can comprise a notch filter, an edge filter and/or a band-pass filter to attenuate any portion of the incident light beam 5107 that may be received at the optical receiver system 5113.

The one or more photodetectors can comprise photodiodes sensitive to light in a wavelength range between 700 nm and 1.5 micron and/or photo multiplier tubes (PMTs). In various embodiments, the optical receiver system 5113 can comprise a single broadband photodetector. In such embodiments, a series of bandpass optical filters can be disposed in the receive path before the single broadband photodetector to transmit light in different wavelength regions. In some embodiments, the optical receiver system 5113 can comprise a photodetector that is sensitive to wavelengths each of the plurality of filtered spectral regions. In such embodiments, each optical bandpass filter can be associated with the photodetector that is sensitive to the light transmitted through that optical bandpass filter. In various embodiments, the optical receiver system 5113 can comprise a Raman spectra appropriate spectrograph, such as, for example, a holographic grating.

The Raman spectra can include peaks associated with the constituents of the tooth 5109 as well as peaks associated with morphological changes in the molecular structure of the constituents of the tooth 5109. Thus the Raman spectra can provide valuable information regarding not only the constituents of the tooth but also associated with the diseased state of the tooth.

The spectral information obtained by the optical receiver system 5113 can be analyzed using an electronic processing system 5115 as discussed above. Without any loss of generality, the electronic processing system 5115 can be configured to analyze the peaks at wavenumbers in a range between about 420 cm⁻¹ and about 450 cm⁻¹ (e.g., 431 cm⁻¹), between about 570 cm⁻¹ and about 610 cm⁻¹ (e.g., 590 cm⁻¹), between about 940 cm⁻¹ and about 980 cm⁻¹ (e.g., 959 cm⁻¹) and/or between about 1020 cm⁻¹ and about 1065 cm⁻¹ (e.g., 1043 cm⁻¹) of the spectral information obtained by the optical receiver system 5113 to examine a tooth or a portion thereof. These peaks can correspond to excitation of the compound PO₄³⁻ which is a constituent of the enamel to higher vibrational/rotational energy levels.

In various implementations, the electronic processing system 5115 can be configured to analyze the ratio of the intensities (e.g., maximum intensities) of a peak in a range between a wavenumber of about 420 cm⁻¹ and a wavenumber of about 450 cm⁻¹ (e.g., 430 cm⁻¹) and a peak in a range between a wavenumber of about 2920 cm⁻¹ and a wavenumber of about 2960 cm⁻¹ (e.g., 2941 cm⁻¹). These peaks can be associated with bending modes of the molecules of the constituent of enamel.

In various implementations, the electronic processing system 5115 can be configured to analyze the ratio of the intensities (e.g., maximum intensities) of a peak in a range between a wavenumber of about 940 cm⁻¹ and a wavenumber of about 980 cm⁻¹ (e.g., 960 cm⁻¹) and a peak in a range between a wavenumber of about 2920 cm⁻¹ and a wavenumber of about 2960 cm⁻¹ (e.g., 2941 cm⁻¹). These peaks can be associated with stretching modes of the molecules of the constituent of enamel.

The ratio of the intensities (e.g., maximum intensities) of peaks at a wavenumber of 430 cm⁻¹ and at a wavenumber of 2941 cm⁻¹ and the ratio of the intensities of peaks at a wavenumber of 960 cm⁻¹ and at a wavenumber of 2941 cm⁻¹ can be representative of a mineralization index of the enamel. For example, a first value of the ratio of the intensities (e.g., maximum intensities) of peaks at a wavenumber of 430 cm⁻¹ and at a wavenumber of 2941 cm⁻¹ can be representative of a first mineralization index and a second value of the ratio of the intensities (e.g., maximum intensities) of peaks at a wavenumber of 430 cm⁻¹ and at a wavenumber of 2941 cm⁻¹ can be representative of a second mineralization index. Similarly, a first value of the ratio of the intensities (e.g., maximum intensities) of peaks at a wavenumber of 960 cm⁻¹ and at a wavenumber of 2941 cm⁻¹ can be representative of a third mineralization index and a second value of the ratio of the intensities (e.g., maximum intensities) of peaks at a wavenumber of 960 cm⁻¹ and at a wavenumber of 2941 cm⁻¹ can be representative of a third mineralization index.

Other metrics can be calculated from the Raman spectra in addition to or instead of peak locations and the ratio of the intensities of the peaks. These other metrics may be representative of the constituents of the tooth and/or changes in the morphology of the constituents of the tooth.

The electronic processing system 5115 can be configured to employ statistical analysis techniques (e.g., principal component analysis, factor analysis, hierarchical cluster analysis, linear discriminant analysis or analysis of variance) on the obtained Raman spectra. In some embodiments, the electronic processing system 5115 can be configured to employ spectral processing such as smoothing of spectral peaks via digital filtering, such as, for example, Savitsky-Golay or a moving average method. In various embodiments, the electronic processing system 5115 can be configured to increase the signal-to-noise ratio of the obtained Raman spectra. For example the electronic processing system 5115 can be configured to increase the signal-to-noise ratio of the obtained Raman spectra by taking multiple acquisitions per spot and averaging the result. As another example, the electronic processing system 5115 can be configured to increase the signal-to-noise ratio of the obtained Raman spectra by subtracting a background to improve the signal-to-noise ratio.

The optical system 5100 can comprise one or more optical fibers. For example, the conditioning optics 5103 can comprise optical fiber based mirrors, filters and/or lenses. As another example, the optical probe 5105 can comprise an optical fiber. In such implementations, one or more optical elements can be included at the output of the optical fiber to manipulate the diverging light exiting from the optical fiber and converge it to create an illumination spot on at least a portion of the tooth and/or to collect the scattered radiation from at least a portion of the tooth. The optical fibers used in the optical system 5100 can have an inner diameter between about 1 micron and about 1 mm and an outer diameter between about 5 micron and about 1.5 mm. The optical fibers used in the optical system 5100 can be single mode fiber or multimode fiber. Various embodiments of optical fibers employed in the optical system 5100 can be coated with a material to provide flexibility and abrasion resistance. Various embodiments of the optical system 5100 can comprise polarization maintaining fibers.

Various embodiments of the optical system 5100 can be configured as a point-based scanning system. An incident beam 5107 having a spot size between about 5 microns and about 500 microns can be generated using one or more optical elements (e.g., one or more lenses) of the conditioning optics 5103. The spot size of the incident beam 5107 can be based upon imaging resolution requirements. It is noted that the surface of the tooth can be non-uniform. The non-uniformity of the tooth surface can change the focal distance of the incident beam 5107. Accordingly, the spot size can change as the beam is scanned from one location to the next. The non-uniformity of the surface of the tooth can also change the distance between the output of the conditioning optics 5103 and the tooth surface. This can change the beam diameter of the light beam 5107.

The conditioning optics 5103 can comprise one or more dynamic optical components configured to steer the incident beam 5107 to a desired location of at least a portion of the tooth. The one or more dynamic optical components can comprise a galvanometer or a resonant or micro electromechanical (MEMs) mirror system. The one or more dynamic optical components can be controlled by the electronic control system to scan different portions of the tooth and/or gum-line. For example, in one embodiment, the one or more dynamic optical components can be controlled by the electronic control system to generate closely spaced line scans of at least a portion of the tooth or gum-line.

At each scanned location, a measurement can be obtained. The measurement can comprise one or more of locations of the peaks in the Raman spectra and/or ratios of intensities (e.g., maximum or peak intensities) of different peaks in the Raman spectra. After the scanning is completed, there are a series of unique values 1:1 mapped to the spatial location, from which an image can be generated. Different indicators can be associated with the individual measurements or a range of measurements. Different spatial locations of tooth can be associated with the different indicators depending on the corresponding value of the measurement. An image of a tooth in which different spatial locations are indicated by different indicators based on the corresponding value of the measurement can be referred to as a hat map.

Various embodiments of the optical system 5100 can be configured as an imaging system configured to image the entire tooth 5109 including the portion of the gum line surrounding the tooth and/or a selected portion of the tooth 5109 or the gum line. In such embodiments, the conditioning optics 5103 can be configured to generate an incident optical beam 5107 having a larger spot size, such as, for example, a spot size between 1 mm and 10 mm. The optical receiver system 5113 can comprise a charge coupled device (CCD) array or an array of cameras that are configured to image the portion of the tooth 5109 being illuminated by the incident optical beam 5107. The image of the portion of the tooth 5109 being illuminated by the incident optical beam 5107 can comprise a plurality of image pixels corresponding to various regions of the portion of the tooth 5109 being illuminated by the incident optical beam 5107. The electronic processing system 5115 can be configured to employ various algorithm and data processing techniques to assign a color level to each image pixel based on a value of a metric obtained from the Raman spectra of the corresponding region of the portion of the tooth 109 being illuminated. In this manner, the electronic processing system 5115 can generate a “Raman signal” map of the portion of the tooth being examined. The electronic processing system 5115 can be further configured to display the generated Raman signal map on a display device associated with the electronic processing system 5115.

Various embodiments of the optical system 5100 can comprise two optical paths. The illumination beam can be directed along a first optical path towards the tooth or a portion thereof. The illumination beam 5107 can be wide enough to illuminate the desired portion of the tooth. Accordingly, the illumination beam can be wide enough to span across the entire surface of the tooth. The Raman scattered light 5113 is directed along a second optical path toward an optical receiver for image generation. One or more bandpass filters can be disposed in the second optical path to collect light in one or more selected range of wavenumbers. The acquisition can be synchronized so that the one or more bandpass filters are sequentially deployed and multiple images collected, each corresponding to Raman-shifted light in a different range of wavenumbers. These individual wavenumber images could then be overlayed onto each other to create a multispectral or hyperspectral (depending on the number of wavenumber ranges) image.

Endogenous dental species, such as enamel, can be birefringent. Thus, the index of refraction of the endogenous dental species, such as enamel, can be polarization dependent. Without subscribing to any particular theory, the birefringence can be attributed to the tight, preferred orientation of the enamel rods within the hydroxyapatite matrix. Demineralization can reduce this level of alignment, which can be detected by a polarization-sensitive method. Accordingly, various embodiments of the optical system 5100 can be configured to be sensitive to polarization. For example, the optical system 5100 can comprise one or more polarization altering optical components (e.g., half or quarter wave plates, retarders, polarizers, or combinations thereof) at different locations in the transmit path and the receive path. The one or more polarization altering optical components can manipulate the excitation light and select Raman scattered photons based on their polarization.

As discussed above, the optical system 5100 can be configured to be mounted on a track that facilitates positioning the optical system 5100 with respect to one or more teeth of the patient. In some embodiments, the optical system 5100 could be contactless and placed in the mouth above a desired location and mechanically held in place via an articulating arm. The arm can be operated remotely or directly by a user (e.g., a dentist or a dentist's assistant) for desired placement with respect to a tooth. The placement could be achieved manually by the user using a joystick or similar function to adjust the optical system's position via control of mechanical motion linear stages. Alternatively, the placement could be automated by the user selecting the desired tooth for measurement and the position adjusted via a feedback between a camera identifying the current position and the mechanical motion devices. In another alternative, the user can position the arm using a camera system that provides feedback regarding the placement of the arm.

In conclusion a Raman spectroscopic system configured to perform dental exam is described herein. The Raman spectroscopic system can comprise a scanning system that is configured to generate a Raman map of the patient's teeth. Embodiments of the Raman spectroscopic system described herein can be configured to form an image of the patient's teeth with different portions of the patient's teeth associated with a metric obtained from the Raman spectra.

### Optical Coherence Tomography

Various optical systems and methods to examine a tooth contemplated by this application comprise an optical coherence tomography (OCT) system. The OCT system is an interferometry-based imaging technique. This application contemplates the use of OCT for a variety of dental applications including but not limited to the detection of caries, identification of tooth defects such as cracks, as a tool for measuring the true root canal working length, to characterize the structure and architecture of inner root canal wall structure, for the visualization and diagnosis of peridontium pathologies, and/or visualization of the apical region of a root canal

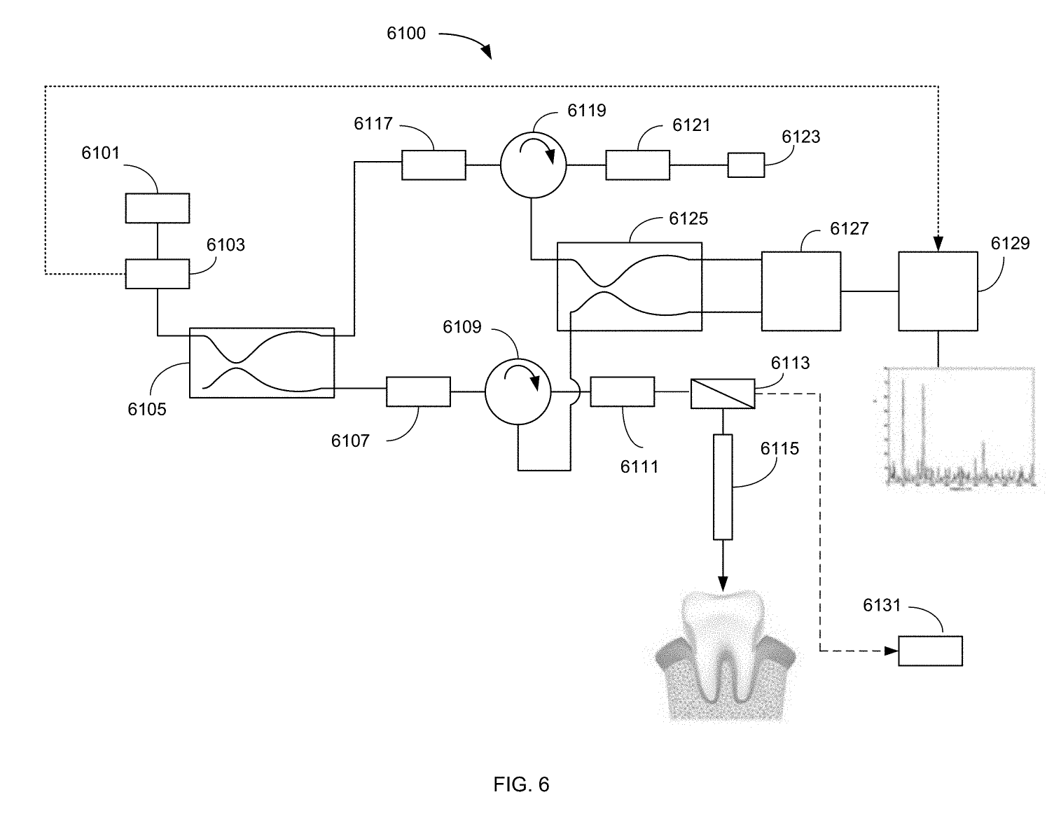

FIG. 6 illustrates an embodiment of an OCT system 6100. The OCT system 6100 comprises an optical source 6101. The light from the optical source 6101 can be divided between a signal arm and a reference arm using an optical splitter 6105 (e.g., a 90-10 splitter). Light from the signal arm of the optical splitter 6105 (e.g., the output of the optical splitter through which 90% of the input light is emitted) is input into a first port of an optical circulator 6109 and output through a second port of the optical circulator 6109. Light output from the second port of the circulator 6109 is directed towards one or more teeth to be examined along a transmit path comprising a collimator 6111, a beam splitter 6113 and an optical train 6115. In various embodiments, the beam splitter 6113 can comprise a dichroic mirror. The optical train 6115 can comprise a plurality of optical components, such as, for example, one or more focusing lenses, one or more collimating lenses, one or more beam steering optical components or combinations thereof. The beam splitter 6113 is configured to direct a first portion of the light reflected or scattered from the one or more teeth to a video camera 6131 to assist the user (e.g., a dentist or a dentist's assistant) to direct the optical signal to a desired location on the one or more teeth. A second portion of the reflected or scattered light from the one or more teeth is directed by the beam splitter 6113 towards the second port of the optical circulator 6109 through the collimator 6111. Light received at the second port of the optical circulator 6109 is output through a third port of the optical circulator 6109 and input into a first arm of a second optical coupler 6125.

Light from the signal arm of the optical splitter 6105 (e.g., the output of the optical splitter through which 10% of the input light is emitted) is input into a first port of an optical circulator 6119 and output through a second port of the optical circulator 6119 towards a reflector 6123 via a collimator 6121. Light reflected from the reflector 6123 is input into the second port of the optical circulator 6119 and output through a third port of the optical circulator 6119 and coupled into a second arm of the second coupler 6125. The optical signal from the second coupler 6125 is provided as an input to a balanced optical detector 6127. The electrical signal output from the balanced optical detector 6127 can be acquired by a high-speed digital acquisition (DAQ) system 6129 to obtain the OCT signal. Polarization controllers 6107 and 6117 can be provided at the output of the signal arm and the reference arm respectively.

Without any loss of generality, the OCT signal is generated by the optical interference of the optical signal reflected/scattered by the one or more teeth and the optical signal reflected from the reflector 6123. The interference condition can be varied by varying the difference in the optical path length between the optical signal reflected or scattered by the one or more teeth and the optical signal reflected from the reflector 6123. In some embodiments, the interference condition can be varied by moving the reflector 6123. In some other embodiments, interference condition can be varied by varying the wavelength of light emitted from the optical source 6101 with time. Implementations of an OCT system in which the wavelength of light output from the optical source 6101 can be referred to as swept source (SS) OCT.

The OCT system 6100 depicted in FIG. 6 is configured as SS-OCT system. Accordingly, the optical source 6101 can comprise a tunable laser or a tunable laser diode which can be tuned over a plurality of discrete wavelengths over time. The SS-OCT system depicted in FIG. 6 can comprise a k-clock 6103 that provides a clock output whose frequency is proportional to the wavelength output by the optical source 6101. The output of the k-clock 6103 can be used as a sampling clock for the DAQ 6129. In SS-OCT systems, the wavelength of the optical source can be tuned to a plurality of discrete wavelengths to generate a spectral scan. As spectral information is not collected simultaneously, there is no need for a spectrometer. Accordingly, SS-OCT systems can be compact as compared to other OCT systems.

The OCT systems that can be used for dental application including the SS-OCT system depicted in FIG. 6 can be optical fiber based, including the k-clock, reference and sample circulators 6119 and 6109 respectively and the balanced detector 6127. The reference collimation arm comprising the collimator 6121 and the signal arm comprising the optical train 6115 can be configured as free space sections to facilitate scanning across the surface of the one or more teeth and to focus light onto a surface of the one or more teeth.

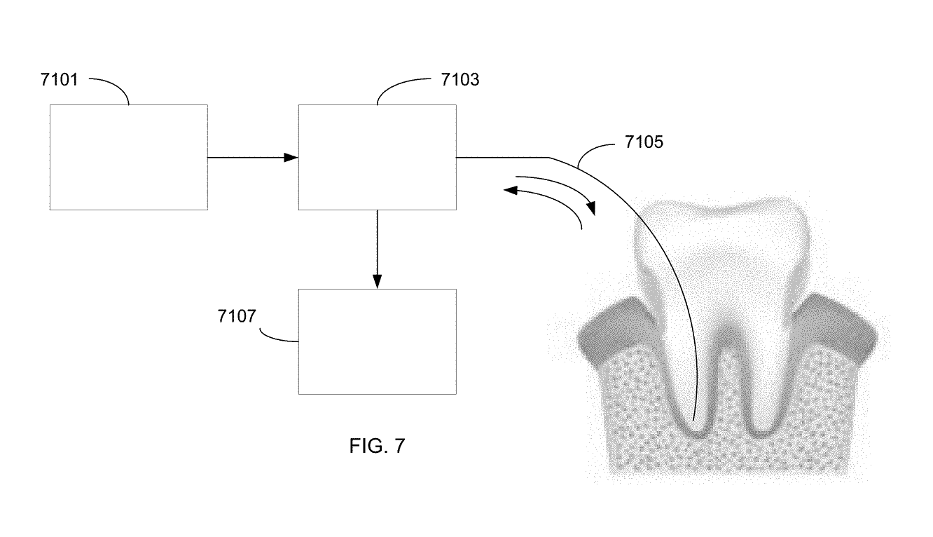

In some embodiments, the OCT system can be configured as a fully fiber-based system as shown in FIG. 7. The fiber-based OCT system can comprise a fiber configured to be inserted into a root canal, with lateral (radial) illumination yielding cross-sectional images (in the radial-axial plane) of the surfaces of the canal. In FIG. 7, light from the optical source 7101 is output from an optical system 7103. In various embodiments, the optical system 7103 can comprise optical components that form the reference arm and the signal arm of the OCT system. Light from the optical system 7103 is input into an optical fiber 7105 that can be inserted into a root canal. Light received from the root canal is input to an optical detector included in the optical system 7103. In some embodiments, the light received from the root canal can be input to the optical detector along with light from the reference arm of the OCT system. The output of the optical detector can be analyzed by an electronic processing system 7107 to image to obtain an image (e.g., an OCT image) of the root canal. For example, a cross-sectional image of the root canal showing the different layers/strata of the root canal (e.g., biofilm, dentin, enamel, cementum, etc.) can be obtained. Different (e.g., each) cross-sectional images can be obtained by scanning the laser along the inner canal wall for a certain length. A cross-sectional, panoramic image can be formed by stitching together these individual images. A length of the root canal (e.g., a working length of the root canal) can be measured or estimated from the one or more cross-sectional images and/or the panoramic image. Additionally, information regarding the morphology of the various layers of the root canal can be obtained from the one or more cross-sectional images and/or the panoramic image. Additionally, for each axial length segment, the laser could be scanned to provide images in the circumferential/azimuthal direction such that a 3D “down-the-pipe” view is obtained.

The generation of cross-section, panoramic images can be used to calculate the working length of the canal and the geometry of the apex including the apical constriction and apical foramen diameters.

The optical source 6101 or the optical source 7101 can be configured to output different excitation wavelengths depending on the tissue of interest. For example, wavelengths in the range between about 1500 nm and about 1600 nm can be used to image hard tissue (enamel, dentin, bone) while wavelength in the range between about 750 nm and about 1000 nm can be used to image soft oral tissue (mucosa or gingiva) so that the light is not absorbed by water or blood. Accordingly, in various implementations, the optical source 6101 or the optical source 7101 can be configured to output wavelengths in a range between about 1500 nm and about 1600 nm.

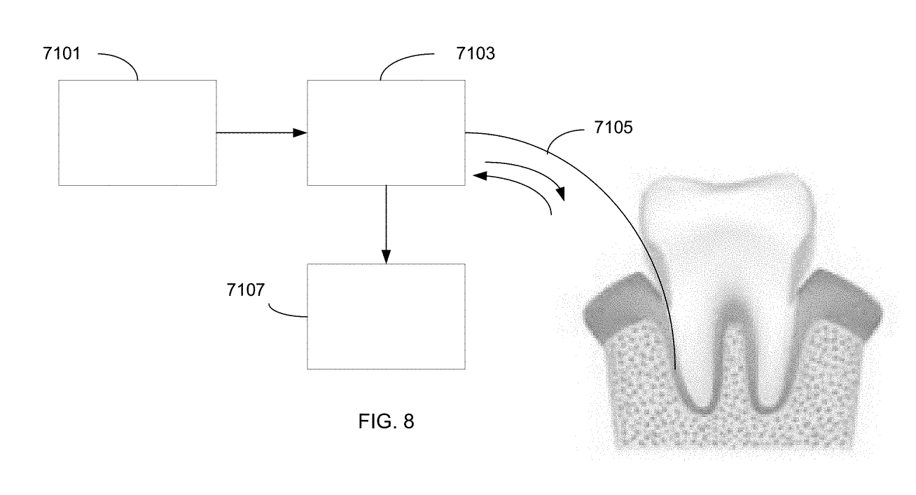

In another fiber-based embodiment, the fiber can be configured to be inserted into a periodontal space, as shown in FIG. 8, to visualize the periodontal space by scanning the external gingival surface. The alveolar bone can be imaged, and in the areas of the mouth where bone is not too thick, the OCT system can be used to visualize the apex itself. In various embodiments, image processing algorithms can be implemented to analyze the OCT images for biological feature such as cracks and caries.

An OCT system can be used in combination with a mechanical device for analyzing a plurality of teeth, for example the scanning mechanical devices described herein. An OCT system in combination with such a mechanical device can provide for measurement and analysis of all tooth surfaces (e.g., occlusal, buccal, inter-proximal, lingual, palatial) and/or of all types of fully erupted or partially erupted teeth (e.g., incisors, pre-molars, molars, 3rd molars). In some embodiments, a scanning device can be used to create a 2D pattern. The scanning device can comprise a galvanometer or a MEMs mirror. An OCT system in combination with a mechanical device for analyzing a plurality of teeth can provide one or more of the following: a depth-of-field ranging from 50 microns to 4 mm, a lateral resolution (in enamel) ranging from 2-12 microns, and an axial resolution (in enamel) ranging from 2-60 microns. In some embodiments, a swept source laser using FDML (Fourier domain mode locking) laser, vertical cavity semiconductor laser (VCSEL) or micro-electro mechanical systems (MEMs) based technologies can be used. In some embodiments, to account for a possible non-linear scanning operation, algorithms capable of “de-warping” the distortion created by non-linear scanning can be used. In some embodiments, a visual imaging system can be used to assist with visual alignment. Such an imaging system can include a camera, an achromatic lens system, dichroic components, or any other imaging system components known in the art. In some embodiments, an OCT system in combination with a mechanical device for analyzing a plurality of teeth can include one or more features for depth adjustment, such as a zoom lens system, to account for varying tissue depths of interrogation. In some embodiments, an OCT system in combination with a mechanical device for analyzing a plurality of teeth can be configured to generate a voxel volume of a tooth structure in under 5 seconds. In some embodiments, the visual imaging system can have the following features: distortion of less than 1% and field of view between 6 mm to 12 mm. In some embodiments, the imaging system can be diffraction limited or near diffraction limited (for example, 0.5 to 3 waves of PV wavefront error) across wavelengths in the visible range, such as, for example, between 380 nm and 700 nm.

The above-described OCT system is configured to detect caries, cavities and/or tooth decays. The OCT system described herein is configured to obtain an OCT image of the morphology of various constituents of the patient's teeth and determine one or more characteristics representative of a condition of the patient's teeth based on an analysis of the OCT image. For example, information regarding the thickness and/or morphology of various layers/strata of the teeth and/or gums can be obtained from the OCT image. As another example, information regarding dimensions (e.g., length, width and/or depth) of carious lesions or cracks in the enamel of the tooth can be obtained from the OCT image. As another example, information regarding depth of a periodontal pocket can be obtained from the OCT image. In some embodiments, information regarding the bacterial load in the periodontal pocket can be obtained from the OCT image. For example, the OCT system can be combined with the fluorescence spectroscopy system to obtain an OCT image of the periodontal region or the root canal as well as estimate the bacterial load in the periodontal region or the root canal. As yet another example, information regarding the length or working length of a root canal can be obtained from the OCT image. The OCT system can be configured to obtain an OCT image of the periodontal space and determine morphology of the periodontal space based on an analysis of the OCT image.

### Light Scattering Based Techniques

This application contemplates optical systems and methods that can spatially quantify the scattering properties across a dental surface to identify carious lesions or tooth defects.

### Spatial Frequency Domain Imaging

The level of scattering and absorption of a surface of a tooth or gum can be determined after mathematically analyzing the collected back-scattered signal from the surface of a tooth or gum illuminated with a structural pattern, in a technique known as spatial frequency domain imaging (SFDI). An optical system configured for SFDI comprises a light source, a spatial light modulator and focusing optics on the delivery side and an imaging system on the collection side. SFDI comprises projecting a sinusoidal pattern onto a surface of a tooth or gum at multiple phases for different wavelengths of light emitted by the illumination source. The illumination source can comprise a laser or an LED. To illuminate the sample at different wavelengths, multiple LEDs or lasers can be used or alternatively a white light source coupled with a tunable optical filter can be used.

As hard dental tissue will have significant specular reflection, crossed linear polarizers can be disposed on the collection side prior to the imaging surface to reject the specular light and collect only those photons that have experienced diffuse scattering events within the tissue. The collection of specular reflection can also be reduced by illuminating at a slightly off-axis (to the vertical). To account for the topography of the measured dental surfaces, height profile data can be determined via illumination at a single spatial frequency.

The penetration depth can be altered by varying the spatial frequency, providing the capability of selective depth sampling. In one embodiment, each dental imaged area is illuminated with two spatial frequencies: one to measure subsurface, superficial layers and the other to measure deeper layers.

Enamel structure comprises enamel rods, which include densely packed, highly orientated hydroxyapatite crystals. The enamel rods are cylindrical in shape and each rod is orientated roughly perpendicular to the dentin enamel junction at which it terminates. This can result in any section of enamel having very homogeneous enamel structural orientation and alignment. This anisotropy of the enamel rods results in an optical property known as birefringence, whereby the index of refraction is higher in the axial direction than along the cross section. Specifically, the birefringence of enamel rods is known as a birefringence of form and is exhibited by many other biological tissues including collagen, the cornea, retina and bone. The birefringent properties of a material can be exploited by modifying the polarization of incident light. For example, light linearly polarized parallel to the enamel rod major axis will have a higher index of refraction (slower phase velocity) than light linearly polarized orthogonal to the major axis (higher phase velocity).

Accordingly, for dental applications, this application contemplates combining SFDI with polarized incident light to exploit the birefringence of enamel and identify the presence of any effect that would cause the “scrambling” of the highly aligned enamel rods such as caries or cracks. An optical system that combines SFDI with polarized incident light (PSFDI) can comprise an illumination source; a digital micromirror device (DMD) that is configured to spatially modulate the incident light into sinusoidal patterns; an optical system to project the structured illumination onto the surface of the tooth or the gum; a polarizer configured to alter the polarization state of light prior to being projected onto the surface of the tooth or the gum; and an imaging system. The polarizer can comprise a linear polarizer mounted on a rotational stage. The polarization state of the spatially modulated incident light can be changed before being projected onto the surface of the tooth or the gum. Light reflected from the surface of the tooth or the gum can pass through a polarizer (e.g., linear polarizer) before being imaged by the imaging system. The imaging system can comprise a camera. The polarization angle of the spatially modulated light can be changed from 0 to 180 degrees at discrete intervals (for example, 5-degree steps) and at each polarization angle, the imaging system can be configured to obtain an image for each polarization state. The obtained images can be analyzed using an electronic processing system to diagnose a disease state of the teeth or the gum.

### Reflectance Imaging

Scattering properties of dental tissue can be measured using a technique known as reflectance spectroscopy. For enamel, the wavelength-dependent reduced scattering coefficient can be described by a power law relationship:

$${μ}_{S}^{′} = {A ( \frac{λ}{{λ}_{0}} )}^{- B} \qquad ( 5 )$$

Where λ₀ is a reference wavelength, A is proportional to the density of the enamel, B is the exponent that is empirically determined and related to the size of the enamel rods. The reduced scattering coefficient can be a metric for caries identification as demineralization will reduce enamel density. Thus, there can be significant differences in values of A and B for different levels of demineralization. For enamel, the scattering coefficient (which is related to the reduced coefficient coefficient), ranges from values of approximately 8 cm⁻¹ to 1-2 cm⁻¹ across the visible wavelength range and is inversely proportional to λ³.

The reduced scattering coefficient is determined by using a calibration look up table that is a database of reflectance spectra for different scattering coefficient values. The database could be empirically measured by collecting a large sample of ex-vivo reflectance spectra measurements from healthy enamel and carious enamel. Alternatively, and more practically, the database is formed by measuring artificial samples, known as phantoms, which have been designed with clinically relevant scattering properties. The reflectance is defined as:

$${R}_{diffuse} ( λ ) = \frac{{I}_{sample} ( λ ) - {I}_{background} ( λ )}{[ {I}_{standard} ( λ ) - {I}_{background} ( λ ) ] \times 100 \text{/} {R}_{standard}} \qquad ( 6 )$$

Where R_{diffuse} is the reflectance, Iₛₐₘₚₗₑ is the raw collected spectra, Ibackground is the background spectrum without the light source on (spectra due to background light and the noise on the CCD) and R_{standard} is the reflectance spectra of a calibrated standard.

For the reflectance spectra collected from a location with unknown scattering properties, numerical fitting algorithms (e.g. non-linear least squares) can be used to determine the scattering properties.

An embodiment of an optical system configured to obtain reflectance spectra of a portion of the tooth can comprise an optical source (e.g., a white light illumination source), an optical system for focusing the light from the optical source into a fiber-optic cable; collection of the reflected light by a multi-fiber bundle; and coupling of the fiber bundle into a spectrometer from which the reflectance spectra can be calculated using the reflectance equation (6) above.

The delivery and collection cables can be included with a single fiber-optic probe. The arrangement of the collection fibers relative to the delivery fiber can take any suitable form; however, the most space efficient configuration is the delivery fiber located at the center with the collection fibers surrounding it in a circular pattern, or series of circles depending on the number of collection fibers used. In another embodiment, spectroscopy can be combined with imaging functionality (e.g., spectral imaging (SI)), which can provide several advantages including but not limited to high spatial and spectral resolution.

The device for spectral imaging can be configured as either a color imaging system or a spectral imaging system, which are methods for combining spectral and spatial information.

Depending on the number of spectral data points required, the system can either be multi-spectral imaging (MSI) with 5-20 spectral bands or hyper-spectral imaging (HSI) with 100+ spectral bands. For spectral imaging, a spectral cube or stack of data can be generated, with 2 spatial dimensions and 1 spectral dimension. Therefore, each element in the cube corresponds to a particular spatial location at a certain spectral band.

For the case of spectral scanning, the imaging wavelength can be chosen by mechanically moving filters where each filter corresponds to one of the spectral bands, for example a rotating disk containing different filters and the rotational speed of the filter is synchronized with the spatial scanning protocol. In another embodiment, the wavelength selection can be achieved with non-moving optical components that are controlled by voltage or acoustic signals such as liquid crystal tunable filters, acousto-optic tunable filters and liquid crystal Fabry-Perot filters.

In another embodiment, the 3D spectral cube can be formed without any spectral or spatial scanning by using, optical components, such as, for example dichroic or polychroic optics (a color camera), multiple Wollastone prism stages, multi-aperture systems utilizing a microlens array with bandpass filters in front of each microlens, computed tomography imaging spectrometer method, and/or coded apertures.

### Visual Display

The various optical systems described above (e.g., 1100, 5100, 6100) can be in communication with a display device. The display device may comprise a monitor or a touch screen. The electronic processing system associated with the various optical systems described above can be configured to process the data obtained from optical interrogation or examination of the teeth using one or more of the optical techniques discussed above, such as, for example, fluorescence spectroscopy, Raman spectroscopy, OCT, SFDI/PSFDI, reflectance spectroscopy, hyper-spectral and/or multi-spectral imaging and generate “heat maps” of diagnostically relevant parameters obtained by a particular technique which can be displayed visually as a “heat map” on the display device. For example, a metric associated with the cleanliness of the root canal estimated from fluorescence spectra obtained at various locations along the length of the root canal can be displayed as a heat map. As another example, the bacterial load estimated from the fluorescence spectra obtained at various locations along the length of the root canal can be displayed as a heat map. As another example, the peak locations, peak intensities, ratios of maximum or peak intensities, Raman indices and/or reduced scattering coefficients at selected wavelengths obtained from the Raman spectra of a tooth or a portion of the tooth can be displayed as a heat map. As yet another example, spatial identification of different morphological regions such as a carious lesion, enamel, dentin, cementum, bone, biofilm, dentin mud etc. obtained from an OCT image can be displayed as a heat map. As another example, automated quantification of regional geometry such as thickness (i.e. enamel or biofilm thickness), depth (lesion depth into enamel or periodontal pocket depth or crack depth), length (working length) or width (crack width) obtained from analyzing the OCT images can be displayed as a heat map.

Without any loss of generality, the heat map can be generated via calibration of a diagnostic metric or a qualitative category to a legend (e.g., a color-coded legend). For example, different values of a diagnostic metric can be associated with different indicias/indicators. The indicators can be a color, a tone level, a texture, etc. Similarly, different qualitative categories can be associate with different indicias/indicators. In some embodiments, the diagnostic metric or the qualitative category can be a numerical diagnostic metric including but not limited to, Raman peak intensities or Raman indices or reduced scattering coefficients at selected wavelengths. In some embodiments, the diagnostic metric or the qualitative category can be associated with the cleanliness of the root canal. In some embodiments, the diagnostic metric or the qualitative category can be associated with the demineralization index. In some embodiments, the diagnostic metric or the qualitative category can be associated with a thickness, a depth, a length or a width of morphological features of the tooth or gum.

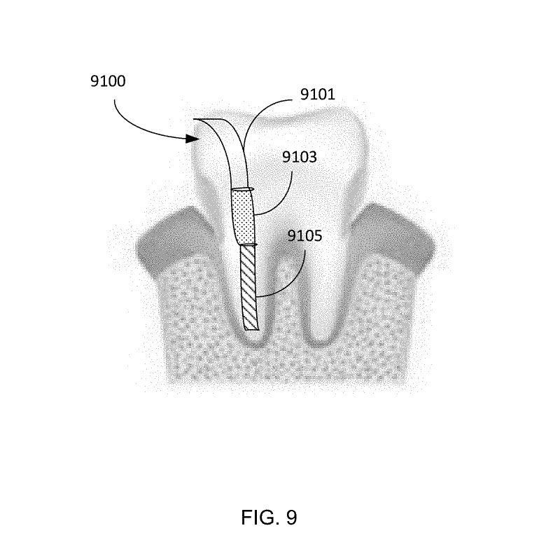

The heat map can be overlaid over an image (e.g., a color image) of the corresponding portion of the tooth or gum that was examined to obtain the data used to generate the heat map. In various embodiments, the image of the examined portion of the tooth or gum can comprise a plurality of image pixels. When overlaying the heat map over the image of the examined portion of the tooth or gum, each element of the heat map can be registered with a corresponding pixel of the image of the examined portion of the tooth or gum which contributed to the data from which that element of the heat map was generated. For example, a heat map of the metric associated with a cleanliness of a root canal can be overlaid over an image of the root canal that was examined as shown in FIG. 9. In FIG. 9, the cleanliness of a root canal 9100 is examined using a fluorescence spectroscopic method as discussed above. The region 9101 of the root canal having low bacterial load/concentration and thus considered to be “clean” can be displayed as green colored region. The region 9103 of the root canal having moderate bacterial load/concentration can be displayed as yellow colored region. The region 9105 having high bacterial load/concentration can be displayed as red colored region. In addition, a remark may be associated with the various regions. For example, an annotation or a remark “monitor” may be associated with the yellow colored region 9103. As another example, an annotation or a remark “immediate treatment” may be associated with the red colored region 9105. In various embodiments, the remark may be displayed along with the heat map. The heat map and/or the remarks/annotations may be configured to allow the dentist/dentist's assistant to easily identify portions of one or more teeth that may need further attention or treatment. In various embodiments, the electronic processing system may be configured to automatically alert the dentist/dentist's assistant in response to detecting caries, cracks, regions of high bacterial load/concentration, or other defects/pathologies. In In some embodiments, the electronic processing system associated with the various optical systems described above and/or the display device may be configured to allow the dentist/dentist's assistant to include remarks associated with the various regions. For example, display system may be configured to allow the dentist/dentist's assistant to select a region (e.g., 9101, 9103, 9105), add a remark associated with that region and/or display the added remark.

The electronic processing system associated with the various optical systems described above (e.g., 1100, 5100, 6100) and/or the display device can be configured to allow the user (e.g., dentist/dentist's assistant) to manipulate the digital display by changing the field of view (zoom in or out) via a touch screen or manual control (keyboard or mouse operation). The electronic processing system associated with the various optical systems described above and/or the display device can be configured to allow the user (e.g., dentist/dentist's assistant) to manipulate the digital display by changing the angle (rotation) via a touch screen or manual control (keyboard, mouse operation or joystick). When a series of teeth are imaged, the electronic processing system associated with the various optical systems described above and/or the display device can be configured to allow the user (e.g., dentist/dentist's assistant) to manipulate the digital display by selecting the tooth of interest.

The generated heat maps, the data associated with the generated heat maps and/or the image of the corresponding portion of the tooth or gum that was examined to obtain the data used to generate the heat map can be stored electronically (e.g., in an electronic memory device) and retrieved at a later stage for visual and/or quantitative comparison. The associated remarks added by the dentist or the dentist's assistant can also be stored electronically.

### Mounting Assemblies

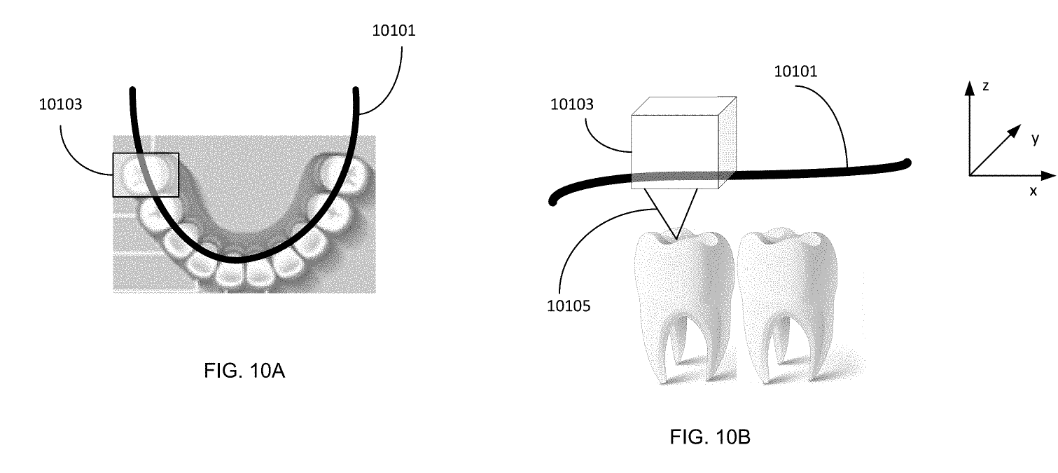

The various optical systems described above or portions thereof can be mounted to a mechanical construction that can be disposed over, adjacent, or otherwise proximate a plurality of teeth. In various embodiments, the mechanical construction can provide a controllable path along which the light delivery system and the light collection systems can be moved to optically examine one or more teeth. In various embodiments, the mechanical construction can comprise a track or a rail. FIGS. 10A-10C depict an example of a housing 10103 comprising an optical assembly. The optical assembly can be a part of any of the optical systems described above that can be mounted to a track 10101. The track 10101 shown in FIG. 10A has a curved shape and overlaps with a plurality of teeth. In various implementations, the track 10101 can comprise a contiguous section covering all mandibular or maxillary teeth. In other embodiments, the track 10101 can comprise a shorter section that covers only a single tooth or a pair of teeth. In some implementations, the track 10101 can be segmented for specific types of teeth (molar, pre-molars or anteriors). The housing 10103 can be slidably mounted to the track 10101 (or otherwise engaged with the track so as to move along a pathway defined by the track 10101) such that the housing 10103 can be moved from one tooth to the next to serially examine various teeth. Accordingly, the housing 10103 can be positioned over a first tooth to be examined and moved to a second tooth to be examined after examination of the first tooth is complete. The housing 10103 can be moved along the track manually by the user (e.g., dentist/dentist's assistant). In some embodiments, the movement of the housing 10103 can be controlled by an automated motion controller which can control the operation of an actuator or motor that moves the housing 10103 along the track 10101. The automated motion controller can be configured to move the housing 10103 in response to signals from the user (e.g., dentist/dentist's assistant). For example, the user (e.g., dentist/dentist's assistant) can control the motion of the housing 10103 using a joystick or buttons. In other embodiments, the automated motion controller can be programmed with a map such that the housing 10103 can automatically move along the track 10101 without user control or intervention.

In various embodiments, the housing 10103 can be mounted on a controllable motion stage attached to the track 10101. The controllable motion stage can be manually controlled or electrically controlled. The controllable motion stage can have multiple degrees of freedom (e.g., linear and rotational degrees of freedom). For example, the controllable motion stage can be configured to move along a direction parallel to the x-axis, y-axis and/or the z-axis (or along the x-y plane, the x-z plane, or the y-z plane). The controllable motion stage can also be configured to be rotated about the x-axis, the y-axis and/or the z-axis. In various embodiments, the controllable motion stage may comprise a gross motion controller and a fine motion controller. The controllable motion stage with the housing 10103 can be positioned over a tooth to be examined using the gross motion controller. The position of the translation stage with the housing 10103 can be locked by a locking mechanism. The position of the interrogation light beam 10105 can be adjusted in the lateral and the vertical directions using the fine motion controller of the translation stage. In some embodiments, the fine motion controller can be used to scan the interrogation light 10105 across the surface of the tooth. As discussed above, the translation stage can be moved manually or using an automated motion controller. In various embodiments, the housing 10103 can be oriented to scan across the occlusal surface, the lingual surface and/or the buccal surface of the tooth to image the outer surface and/or side surfaces of the tooth.

In embodiments of the housing 10103 configured to direct light into a root canal, the housing 10103 can be configured to be moved along the z-axis and/or rotated about the z-axis. For example, in such embodiments, the housing 10103 can be translated up and down along the z-axis to image along the length of the root canals spaces and/or the pulp chamber. In such embodiments, the housing 10103 may be rotated (for example, in embodiments that utilize a side-fire optical fiber) to circumferentially examine the wall(s) of the root canal(s).

In various embodiments, the track 10101 can be supported at the patient's mouth in any suitable manner. For example, in some embodiments, the track 10101 can be integrated with a mouth guard, which can be worn by the patient. The track 10101 can comprise a stand (e.g., a translation stage) to which the housing 10103 can be attached in other embodiments. The stand can comprise one or more locking mechanisms, such as, for example, screws or clips that can be engaged to fix the position of the stand on the track.

The housing 10103 can include an optical assembly comprising one or more components of the various optical systems described above. For example, the 10103 can comprise the delivery optics that deliver the light for optically examining the tooth and the collection optics that collect light reflected, emitted, and/or scattered by the tooth being examined. Other components of the various optical system described above such as the optical source, the optical receiving system, the display device and/or the electronic processing system can be disposed outside the housing 10103 (for example, within a console of the system). Accordingly, as shown in FIG. 10C, the housing 10103 and the track 10101 can be disposed in the mouth of the patient and the optical source 10107 including a dichroic filter and one or more optical components, the optical receiving system 10109 including the optical detectors and the electronic processing system and the display device 10111 can be disposed outside the mouth of the patient.

The housing 10103 can be in optical and/or electrical communication with the optical source 10107 including a dichroic filter and one or more optical components. In various embodiments, the housing 10103 can comprise freespace optical elements that can direct the interrogation light 10105 to a desired portion of the tooth or gum through an opening in the housing 10103. In various embodiments, the housing 10103 can comprise an optical fiber configured to deliver the interrogation light 10105 to a desired portion of the tooth or gum through an opening in the housing 10103.

In various embodiments, the mounting assembly can comprise a first track configured to engage with a lower jaw with a first housing having an opening facing the teeth of the lower jaw that can be used to optically examine teeth of the lower jaw, and a second track configured to engage with an upper jaw with a second housing having an opening facing the teeth of the upper jaw that can be used to optically examine teeth of the upper jaw. In some embodiments, the mounting assembly can comprise a single track that is used to optically examine teeth of the lower jaw and upper jaw. In such embodiments, the housing can be rotated or flipped such that the opening faces the teeth of the upper or lower jaw that are being examined.

In some embodiments, the housing can be positioned in the mouth of the patient without a track. In such embodiments, the housing 10103 can be placed in the mouth above a desired location and mechanically held in place via an articulating arm. The arm can be operated remotely or directly by a user (e.g., a dentist or a dentist's assistant) for desired placement with respect to a tooth. The placement could be achieved manually by the user using a joystick or similar function to adjust the optical system's position via control of mechanical motion linear stages. Alternatively, the placement could be automated by the user (e.g., a dentist or a dentist's assistant) selecting the desired tooth for measurement and the position adjusted via a feedback between a camera identifying the current position and the mechanical motion devices. In another alternative, the user can position the arm using a camera system that provides feedback regarding the placement of the arm.

The optical systems described herein can advantageously allow rapid analysis of a plurality of teeth. The optical systems described herein can facilitate measurement of various tooth surfaces (occlusal, buccal, inter-proximal, lingual, palatial). All types of fully erupted or partially erupted teeth (incisors, pre-molars, molars, 3rd molars) can be examined and/or image using the optical systems and methods described herein. The optical system described herein can comprise a scanning device (e.g., a galvanometer or a MEMs mirror) to create a 2D pattern. Various embodiments of the optical systems described herein can have a depth-of-field ranging from about 50 microns to about 4 mm. Various embodiments of the optical systems described herein can have a lateral resolution (in enamel) ranging from 2-12 microns. Various embodiments of the optical systems described herein can have an axial resolution (in enamel) ranging from 2-60 microns. Various embodiments of the OCT systems described herein can comprise a swept source laser. The swept source laser can include a FDML (Fourier domain mode locking) laser, a vertical cavity semiconductor laser (VCSEL) or a laser based on MEMs technology. Various embodiments of the optical systems described herein can include algorithms capable of “de-warping” the distortion created by non-linear scanning. Various embodiments of the optical systems described herein can include a visual imaging system to help the user (e.g., dentist/dentist's assistant) with visual alignment. The visual imaging system can include components, such as, for example, camera, achromatic lens system and/or dichroic optical element. Various embodiments of the optical systems described herein can include a zoom lens to facilitate optical interrogation at various tissue depths. Various embodiments of the optical systems described herein can be capable of generating a voxel volume of a tooth structure in under 5 seconds. The visual imaging system can be configured to have less than 1% distortion, field of view in the range between 6-12 mm and/or can be diffraction limited or close to diffraction limited. For example, the visual imaging system can have 0.5 to 3 waves of PV wavefront error) across wavelengths in the visible spectral range.

### Examples of Dental Treatment Systems

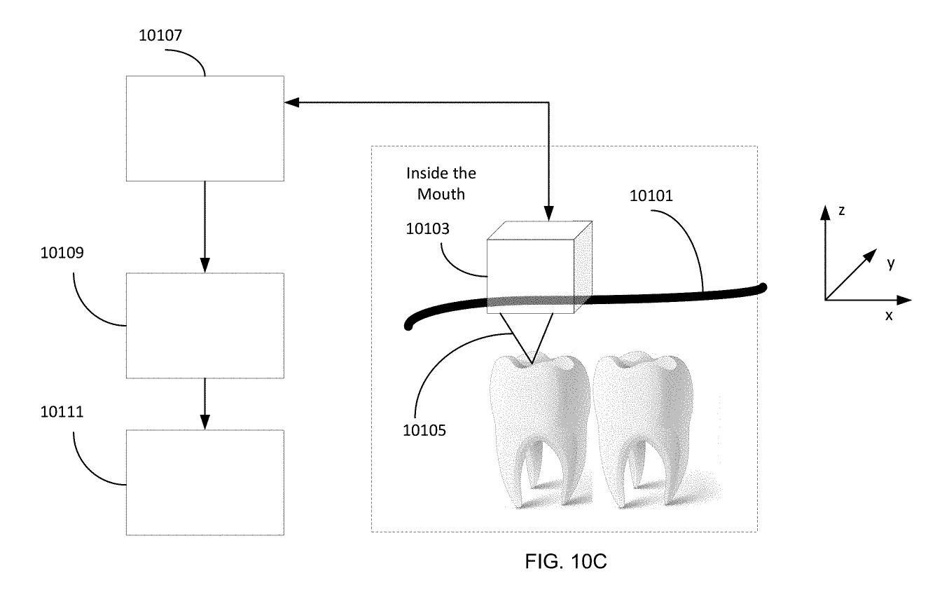

Each of the optical or examination systems and techniques disclosed herein can be used in conjunction with any suitable dental treatment procedure or system. For example, FIG. 11 is a schematic system diagram of a dental system 1. The dental system 1 can comprise a treatment system 2 and an examination system 4. The examination system 4 can comprise one or more optical systems 1100, 5100, 6100 described above. The dental treatment system 2 can be configured to clean a root canal of a tooth, to clean a carious region from an exterior surface of a tooth, to clean gums and periodontal pockets, and/or to clean deposits and plaque from an exterior surface of the tooth. The treatment system 2 can comprise any suitable type of dental treatment system (e.g., a cleaning system, an obturation system, etc.). As explained herein, in various embodiments, the treatment system 2 can comprise a pressure wave generator configured to generate pressure waves at the treatment region having sufficient energy to clean the treatment region, and/or configured to generate fluid motion to improve the removal of material from the treatment region.

The examination system 4 can comprise any of the examination or optical system described herein. In various embodiments, the examination system 4 can be configured to detect a progression of diseased tissue (for example, a carious region on the outer surface of the tooth) before a cleaning procedure is performed. In other embodiments, the examination system 4 can be configured to monitor a cleaning procedure during cleaning of the treatment region (e.g., during the cleaning of a root canal space or a carious region on an external surface of the tooth). In still other embodiments, the examination system 4 can be configured to detect the cleanliness of a treated region (e.g., root canal, exterior surface of the tooth, periodontal pocket, etc.) after a cleaning procedure.

Beneficially, the clinician can utilize the examination system 4 to monitor in real-time whether the monitored region of the tooth is sufficiently clean. Furthermore, the system 1 shown in FIG. 11 can advantageously include both a treatment system 2 and an examination system 4, which can enable the clinician to use a common console for treating and examining or imaging a tooth. In some embodiments, the treatment system 2 and the examination system 4 can be housed in a common console with one or more controllers and a user interface configured to control the operation of the dental system 1. In some embodiments, the one or more controllers of the console can control the operation of both the treatment system 2 and the examination system 4. In other embodiments, however, the treatment system 2 and the examination system 4 can be provided in separate housings or consoles. The treatment system 2 and examination system 4 can be in data communication with one another in various embodiments, such that the treatment system 2 can receive information about the cleanliness of a region of the tooth.

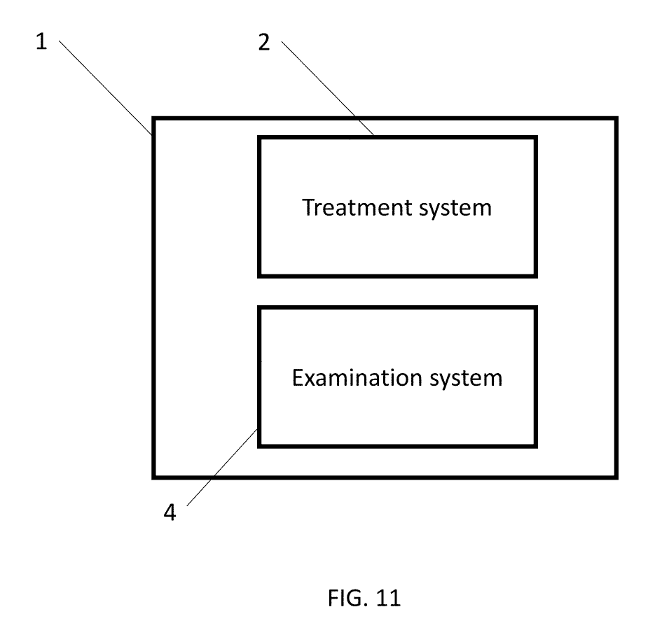

FIG. 12 schematically illustrates an example of a treatment system 2 for treating (e.g., cleaning) a tooth 10 with a pressure wave generator 64, according to various embodiments. The treatment system 2 of FIG. 12 is configured to clean a root canal 30 of the tooth 10. The tooth 10 shown in FIG. 12 comprises a molar tooth, but the system 2 can treat other types of teeth, such as pre-molars, anteriors, etc. An endodontic access opening can be formed into the tooth 10, for example, on an occlusal surface, a buccal surface, or a lingual surface. The access opening provides access to a portion of the pulp cavity 26 of the tooth 10. The system can include a fluid retainer 66 and the pressure wave generator 64. The pressure wave generator 64 can be electrically connected to a source of electrical power by an electrical lead 63.

The fluid retainer 66 can comprise a cap 70 and a flow restrictor 68 that inhibits flow of fluid from the tooth 10. The flow restrictor 68 may also inhibit flow of air into the tooth 10. The cap 70 may be formed from a sufficiently durable, biocompatible substance such as metal or plastic. The flow restrictor 68 may include a sponge, a membrane (permeable or semi-permeable), or a vent. The flow restrictor 68 may limit fluid pressure in the tooth 10 such that if the fluid pressure rises above a threshold, fluid can leak or flow from the tooth chamber through the flow restrictor 68. The use of a flow restrictor 68 advantageously may prevent fluid pressure in the tooth chamber (e.g., in the pulp chamber 28 or at the apex 32 of the tooth) from rising to undesirable or unsafe levels. Fluids as described herein generally means liquids, and the liquids may include a certain amount of dissolved gas. For example, a fluid can include water (having a normal dissolved gas (e.g., air) content as can be determined from Henry's law for the appropriate temperature and pressure conditions) or degassed water, which can have a reduced dissolved gas content as compared to water with a normal dissolved gas content. In various embodiments, for example, the treatment fluid can be substantially degassed, e.g., so as to have less than about 5% dissolved gases by volume, less than about 1% dissolved gases by volume, less than about 0.5% dissolved gases by volume, or less than about 0.1% dissolved gases by volume.

The fluid retainer 66 may include a handpiece (not shown) by which a dental practitioner can apply or maneuver the fluid retainer 66 relative to the tooth 10 during treatment. In some implementations, the fluid retainer 66 can be applied to the tooth with a mechanical clasp or clamp, a dental adhesive, or by pressure applied by the patient by biting on the retainer.

As schematically illustrated in FIG. 12, a distal end of the pressure wave generator 64 can be disposed in the fluid in a tooth chamber 65 in the tooth (sometimes the tooth chamber 65 may be referred to herein as a tooth cavity). The tooth chamber 65 may include at least a portion of any space, opening, or cavity of the tooth 10, including any portion of spaces, openings, or cavities already present in the tooth 10 (either by normal or abnormal dentin and/or tissue structure or by degeneration, deterioration, or damage of such structure) and/or any portion of spaces, openings, or cavities formed by a dental practitioner during a treatment. For example, the tooth chamber 65 may include at least a portion of the pulp chamber 28 and may also include at least a portion of one or more of the following: an access opening to the tooth, a root canal space 30, and a tubule. In some treatments, the tooth chamber 65 can include some or all of the root canal spaces 30, accessory canals, and tubules in the tooth 10. In some treatments, the access opening can be formed apart or separately from the tooth chamber.

The distal end of the pressure wave generator 64 may be disposed in the tooth chamber, for example, in the pulp chamber 28. The distal end of the pressure wave generator 64 may be sized or shaped to fit in the tooth chamber. For example, the distal end of the pressure wave generator may be sized to fit in or through an endodontic access opening formed in the tooth. In some treatment methods, the distal end of the pressure wave generator 64 may be disposed within a few millimeters of the floor of the pulp chamber 28. In other methods, the distal end of the pressure wave generator 64 can be disposed in the fluid retained by the fluid retainer 66, but outside the pulp cavity 26 (e.g., beyond the occlusal surface of the tooth). In some implementations, the pressure wave generator 64 (in addition to or as an alternative to the fluid retainer 66) may be coupled to a handpiece or portable housing that may be maneuvered in the mouth of the patient so as to position or orient the pressure wave generator 64 relative to a desired tooth under treatment.

The distal end of the pressure wave generator 64 may be submerged in fluid in the tooth chamber during at least a portion of the endodontic procedure. For example, the distal end of the pressure wave generator 64 may be disposed in the tooth chamber 65 while there is little or not liquid in the tooth chamber. Fluid can be added to the tooth chamber such that a fluid level rises above the distal end of the generator 64. The pressure wave generator 64 may then be activated for at least a portion of the endodontic procedure. During other portions of the procedure, the generator 64 may be inactive and/or above the fluid level in the tooth chamber 65. Although not shown in FIG. 12, in various embodiments, the fluid platform can comprise a fluid outlet or suction port in fluid communication with a vacuum pump. The vacuum pump can be activated to remove fluid and organic material from the treatment region. In addition, in some embodiments, one or more vents can be provided in the fluid platform and exposed to ambient air. The one or more vents can beneficially be used to regulate the pressure of the system 2.

In various implementations, the pressure wave generator 64 can include a liquid jet device. In some embodiments, the liquid jet device comprises a positioning member (e.g., a guide tube) having a channel or lumen along which or through which a liquid jet can propagate. The distal end portion of the positioning member may include an impingement surface on which the liquid jet impinges and is deflected into jets or spray. The distal end portion of the positioning member may include one or more openings that permit the jet to interact with the fluid in the surrounding environment (e.g., fluid in the tooth chamber) and also permit the deflected liquid to exit the positioning member and interact with the surrounding environment in the tooth 10 (e.g., the tooth chamber and the fluid in the tooth chamber). The result of these interactions can be generation of pressure waves and fluid circulation in the tooth chamber 65, which can at least partially clean the tooth. In some treatment methods, the openings disposed at or near the distal end portion of the positioning member are submerged in fluid retained in the tooth 10 by the fluid retainer 66. In some embodiments the liquid jet device may function as a fluid inlet to the tooth chamber 65 and may deliver fluid to at least partially fill the chamber. Accordingly, in some such embodiments, the liquid jet device functions as a pressure wave generator 64 and as a fluid inlet.

In some embodiments, the pressure wave generator 64 may include a sonic, ultrasonic, or megasonic device (e.g., a sonic, ultrasonic, or megasonic paddle, horn, or piezoelectric transducer), a mechanical stirrer (e.g., a motorized propeller or paddle or rotating/vibrating/pulsating disk or cylinder), an optical system that can provide optical energy to the tooth chamber 65 (e.g., an optical fiber that propagates laser light into the tooth chamber), or any other device that can cause a pressure wave to be generated in the tooth or in a propagation medium in the tooth (e.g., the fluid retained in a tooth chamber).

In some embodiments, the cap 70 is not used. For example, the flow restrictor 68 may be applied to the occlusal surface of the tooth 10 around or over the access opening, and the distal end of the pressure wave generator 64 can be inserted into the tooth chamber 65 through the flow restrictor 68 (or an opening in the flow restrictor).

### Examples of Acoustic Cavitation Produced by the Pressure Wave Generators Disclosed Herein

The pressure wave generator 64 can be configured to generate an acoustic wave 67 that can propagate through the tooth and/or the fluid in the tooth chamber 65 and can detach or dissolve organic and/or inorganic material from dentinal surfaces and/or dissociate pulpal tissue. The fluid in the tooth chamber 65 can act as a propagation medium for the acoustic wave 67 and can help propagate the acoustic wave 67 toward the apex 32 of the root canal space 30, into tubules, and into other spaces in the tooth where organic matter may be found. The acoustic wave 67 may cause or increase the efficacy of various effects that may occur in the tooth 10 including, but not limited to, acoustic cavitation (e.g., cavitation bubble formation and collapse, inertial cavitation, microjet formation), acoustic streaming, microerosion, fluid agitation, fluid circulation, vorticity, sonoporation, sonochemistry, and so forth. The acoustic energy may be sufficient to cause organic and/or inorganic material in the tooth to be detached from surrounding dentin. It is believed (although not required) that the effects caused (or enhanced) by the acoustic energy may lead to a cleaning action that delaminates or detaches the pulpal tissue from the root canal wall, dentinal surfaces, and/or tubules, and may further break such tissue down into smaller pieces.

Without subscribing to or being limited by any particular theory or mode of operation, the acoustic field generated by the pressure wave generator 64 may generate a cavitation cloud within the fluid retained in the tooth chamber 65. The creation and collapse of the cavitation cloud (and/or the jet impacting the impingement surface) may, in some cases, generate a substantial hydroacoustic field in the tooth 10. This acoustic field may generate pressure waves, oscillations, and/or vibrations in or near the canal spaces of the tooth and/or interior dentinal surfaces, which are filled with dentinal tubules. Further cavitation effects may be possible, including growth, oscillation, and collapse of cavitation bubbles formed in or near the tubules (e.g., possibly at the high surface-energy sites of the tubules). These (and/or other) effects may lead to efficient cleaning of the pulp chamber 28 of the tooth.

Additional details of the system 2 shown in FIG. 12 and components thereof may be found throughout U.S. Pat. Nos. 9,675,426 and 9,492,244, the entire contents of each of which are hereby incorporated by reference herein in their entirety and for all purposes.

### Examples of Acoustic Power Generated by Pressure Wave Generators

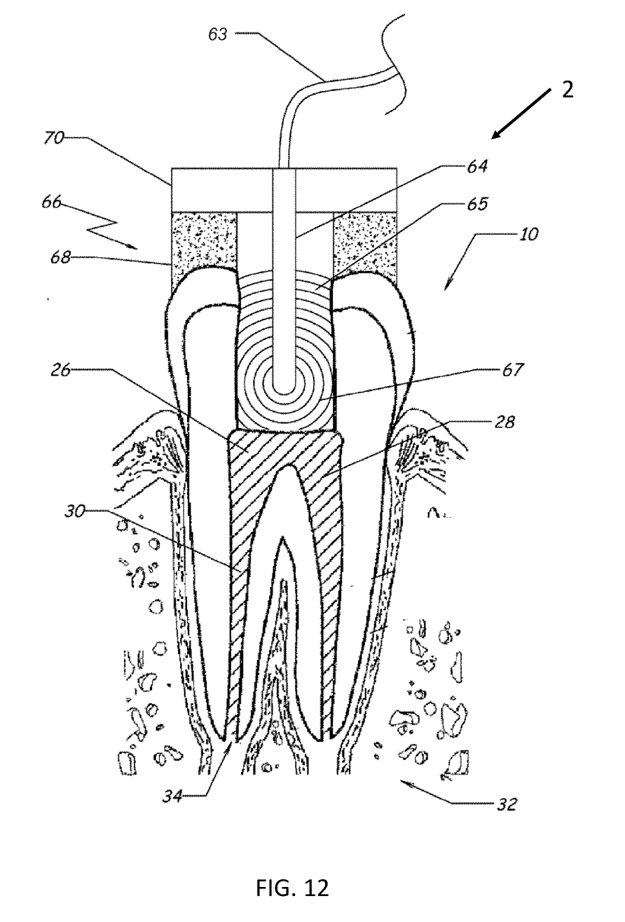

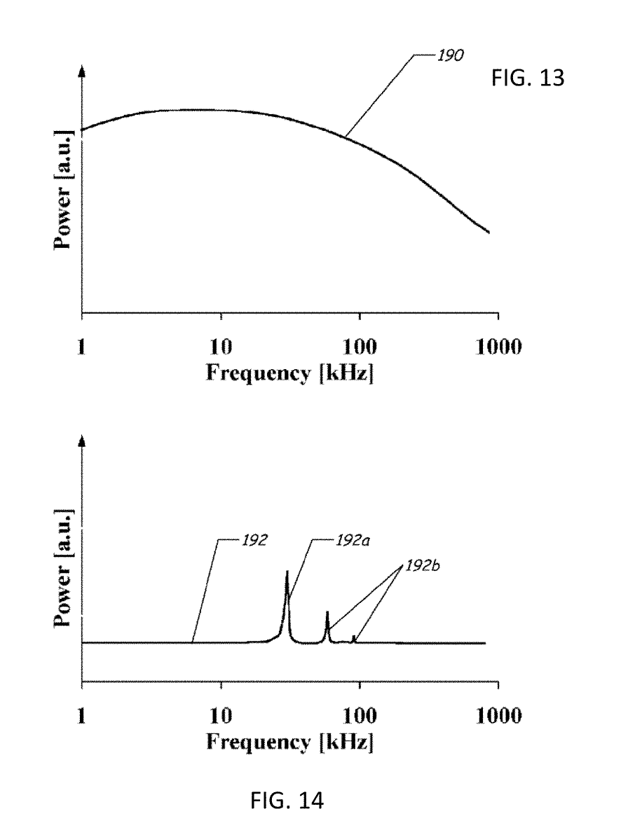

FIGS. 13 and 14 are graphs that schematically illustrate possible examples of acoustic power that could be generated by different embodiments of the pressure wave generators disclosed herein. These graphs schematically show acoustic power (in arbitrary units) on the vertical axis as a function of acoustic frequency (in kHz) on the horizontal axis. The acoustic power in the tooth may influence, cause, or increase the strength of effects including, e.g., acoustic cavitation (e.g., cavitation bubble formation and collapse, microjet formation), acoustic streaming, microerosion, fluid agitation, fluid circulation, sonoporation, sonochemistry, and so forth, which may act to dissociate organic material in the tooth 10 and effectively clean the pulp cavity 26 and/or the canal spaces 30. In various embodiments, the pressure wave generator 64 may produce an acoustic wave 67 including acoustic power (at least) at frequencies above: about 0.5 kHz, about 1 kHz, about 10 kHz, about 20 kHz, about 50 kHz, about 100 kHz, or greater. The acoustic wave 67 may have acoustic power at other frequencies as well (e.g., at frequencies below the aforelisted frequencies).

The graph in FIG. 13 represents a schematic example of acoustic power generated by a liquid jet impacting a surface disposed in a tooth chamber 65 and by the interaction of the liquid jet with fluid in the tooth chamber. This schematic example shows a broadband spectrum 190 of acoustic power with significant power extending from about 1 kHz to about 1000 kHz (e.g., the bandwidth may about 1000 kHz). The bandwidth of the acoustic energy spectrum may, in some cases, be measured in terms of the 3-decibel (3-dB) bandwidth (e.g., the full-width at half-maximum or FWHM of the acoustic power spectrum). In various examples, a broadband acoustic power spectrum may include significant power in a bandwidth in a range from about 1 kHz to about 500 kHz, in a range from about 10 kHz to about 100 kHz, or some other range of frequencies. In some implementations, a broadband spectrum may include acoustic power above about 1 MHz. In some embodiments, the pressure wave generator 64 can produce broadband acoustic power with peak power at about 10 kHz and a bandwidth of about 100 kHz. In various embodiments, the bandwidth of a broadband acoustic power spectrum is greater than about 10 kHz, greater than about 50 kHz, greater than about 100 kHz, greater than about 250 kHz, greater than about 500 kHz, greater than about 1 MHz, or some other value. In some cleaning methods, acoustic power between about 20 kHz and 200 kHz may be particularly effective. The acoustic power may have substantial power at frequencies greater than about 1 kHz, greater than about 10 kHz, greater than about 100 kHz, or greater than about 500 kHz. Substantial power can include, for example, an amount of power that is greater than 10%, greater than 25%, greater than 35%, or greater than 50% of the total acoustic power (e.g., the acoustic power integrated over all frequencies).

The graph in FIG. 14 represents a schematic example of acoustic power generated by an ultrasonic transducer disposed in a tooth chamber 65. This schematic example shows a relatively narrowband spectrum 192 of acoustic power with a highest peak 192a near the fundamental frequency of about 30 kHz and also shows peaks 192b near the first few harmonic frequencies. The bandwidth of the acoustic power near the peak is about 5 to 10 kHz, and can be seen to be much narrower than the bandwidth of the acoustic power schematically illustrated in FIG. 13. In other embodiments, the bandwidth of the acoustic power can be about 1 kHz, about 5 kHz, about 10 kHz, about 20 kHz, about 50 kHz, about 100 kHz, or some other value. The acoustic power of the example spectrum 192 has most of its power at the fundamental frequency and first few harmonics, and therefore the ultrasonic transducer of this example may provide acoustic power at a relatively narrow range of frequencies (e.g., near the fundamental and harmonic frequencies). The acoustic power of the example spectrum 190 exhibits relatively broadband power (with a relatively high bandwidth compared to the spectrum 192), and the example liquid jet may provide acoustic power at significantly more frequencies than the example ultrasonic transducer.

It is believed, although not required, that acoustic waves having broadband acoustic power (see, e.g., the example shown in FIG. 13) may generate cavitation that is more effective at cleaning teeth than cavitation generated by acoustic waves having a narrowband acoustic power spectrum (see, e.g., the example shown in FIG. 14). For example, a broadband spectrum of acoustic power may produce a relatively broad range of bubble sizes in the cavitation cloud, and the implosion of these bubbles may be more effective at disrupting tissue than bubbles having a narrow size range. Relatively broadband acoustic power may also allow acoustic energy to work on a range of length scales, e.g., from the cellular scale up to the tissue scale. Accordingly, pressure wave generators that produce a broadband acoustic power spectrum (e.g., some embodiments of a liquid jet) may be more effective at tooth cleaning for some endodontic treatments than pressure wave generators that produce a narrowband acoustic power spectrum. In some embodiments, multiple narrowband pressure wave generators may be used to produce a relatively broad range of acoustic power. For example, multiple ultrasonic tips, each tuned to produce acoustic power at a different peak frequency, may be used.

### Additional Treatment Systems

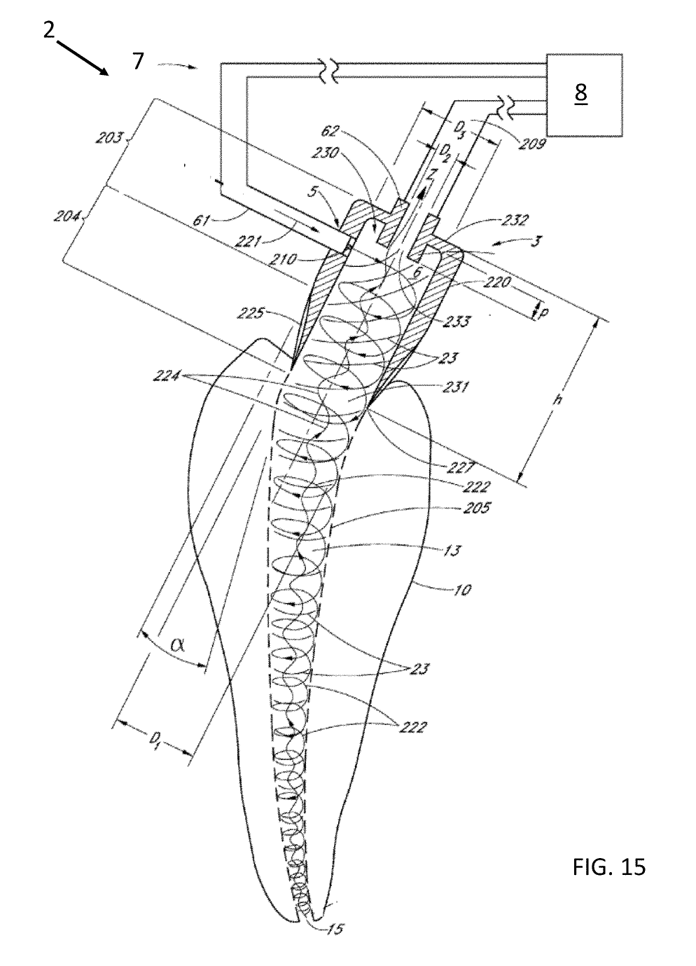

FIG. 15 schematically illustrates an example of a treatment system 2 for treating (e.g., cleaning) a tooth 10 with a fluid motion generator 5 that comprises a pressure wave generator, according to various embodiments. The system 2 shown in FIG. 15 is illustrated during cleaning of a pre-molar or anterior tooth. In other embodiments, the system 2 can be configured to clean a molar tooth.

As shown in FIG. 15, a treatment instrument or cap 3 can be configured to be applied to (e.g., pressed against or attached to) a treatment region of the tooth 10. A fluid motion generator 5 (which may be a pressure wave generator) can be activated to clean the treatment region. The fluid motion generator 5 or pressure wave generator may generate broadband pressure waves, similar to the spectrum shown in FIG. 13. The system 2 can include a console 8 configured to control the operation of the system 2 and one or more conduits 7 that provide fluid communication (and/or electrical or wireless/electronic communication) between the cap 3 and the console 8. The console 8 can include one or more fluid pumps and reservoirs that can supply treatment liquids to the treatment region of the tooth 10. The console 8 can also comprise a fluid removal system including a suction pump and a waste reservoir for removing liquids and waste materials from the tooth 10 by way of the conduit(s) 7. The console 8 can also include one or more processors that are configured to electronically control the operation of the evacuation and/or delivery pumps to control and the delivery of liquid to the tooth and the removal of liquid from the tooth.

The cap 3 can comprise a chamber 6 defined at least in part by an upper wall 232 and a side wall 220 that extends transversely from the upper wall 232. When coupled to the tooth 10 (e.g., pressed against the tooth or attached to the tooth), the chamber 6 can retain liquid and other materials during a treatment procedure. The upper wall 232 and side wall 220 may be integrally formed as a single component in some embodiments; in other embodiments the upper wall 232 and side wall 220 may comprise separate components that are connected or joined together. The side wall 220 can extend annularly relative to the upper wall 232 to at least partially define the chamber 6. It should be appreciated that the upper wall 232, as used herein, refers to the wall near the proximal end of the chamber 6; thus, during some treatments (such as those of upper teeth), the upper wall 232 may be disposed in a downward orientation.

In addition, the cap 3 or chamber 6 can include a distal portion 227 configured to contact the treatment region of the tooth (or a portion thereof). The distal portion 227 can define an access port 231 that provides fluid communication between the chamber 6 and the treatment region of the tooth 10 (e.g., a root canal 13). In various arrangements, the distal portion 227 can taper radially inwardly towards a central axis Z of the cap 3 and/or chamber 6. The central axis Z can be perpendicular to and comprise a central axis of the access port 231. For example, the side wall 220 can comprise a substantially conical taper that continuously and substantially linearly tapers inwardly and distally. Thus, as shown in FIG. 15, a proximal portion of the chamber 6 can have an inner diameter D₃ (or other major dimension) and the access port 231 of the distal portion 227 can have an inner diameter D₁ (or other major dimension) that is smaller than D₃. The chamber 6 may also have a height h. The height h of the chamber 6 can be less than about 5 cm in various embodiments, e.g., less than about 2 cm. Moreover, although not illustrated in FIG. 15, a sealing member can be disposed about the chamber 6 and cap 3. The sealing member can comprise a compressive material (such as a foam) that can seal the treatment region when pressed against the tooth by the clinician. When pressed against the tooth, the cap 3 can be urged into the tooth such that the sealing member is proximal the distal end of the cap 3.

For root canal treatments, as shown in FIG. 15, the distal portion 227 can be inserted into or onto an access opening of the tooth 10 to provide fluid communication with the root canal 13. A sealing material 225 may be applied between the distal portion 227 and the tooth 10 to create or enhance a fluid seal such that liquid, air, and/or debris does not escape to or from the chamber 6 and/or the tooth 10. As shown in FIG. 15, the distal portion 227 can be tapered such that the taper extends from an intermediate or proximal portion of the cap 3 to the distal-most end of the cap 3. For example, as shown in FIG. 15, the side wall 220 of the cap 3 can comprise a generally straight or cylindrical portion 203 (along which the diameter D₃ remains substantially constant) and a tapered or conical portion 204 that tapers inwardly and distally from the straight portion 203 such that the inner diameter D₁ decreases along the distal direction (e.g., towards the tooth 10 in FIG. 15). The tapered portion 204 can be disposed distal the straight portion 203 and can include the distal portion 227 and the distal-most end of the cap 3. Tapering the cap 3 as shown in FIG. 15 can advantageously enable the clinician to conduct treatment procedures on teeth of any size, including very small teeth or teeth that have very small root canal spaces, e.g., the smallest human tooth that would be treated by the system 2. For example, the distal portion 227 can be sized to treat teeth with endodontic access openings having sizes (e.g., diameters or other major dimension) in a range of about 0.5 mm to about 5 mm.

The fluid motion generator 5 (which may also be a pressure wave generator, as described above) can be disposed on and/or through the side wall 220 of the cap 3. The fluid motion generator 5 can be disposed eccentrically relative to a central axis of the cap 3. The fluid motion generator 5 can supply liquid 221 to the chamber 6 so as to generate rotational liquid motion in the chamber 6. The supplied liquid 221 can comprise a degassed liquid as explained herein. The supplied liquid 221 can be any suitable type of treatment fluid, including, e.g., water, EDTA, bleach, obturation material (for filling procedures), etc. For example, a fluid inlet 61 can supply pressurized liquid 221 to the chamber 6. In FIG. 15, the pressurized liquid 221 can be passed through a nozzle 210 at a location in the side wall 220 of the cap 3 (e.g., a sealing cap) at a location near the top wall 232. The nozzle 210 can be disposed offset from the central axis of the chamber 6. Thus, the fluid motion generator 5 may be off-center or asymmetric relative to the cap 3. For example, the fluid inlet 61 and the nozzle 210 can be offset relative to the central axis Z of the cap 3. As shown in FIG. 15, the central axis Z can pass distally along the height h of the cap 3 through the center of the access port 231, e.g., the central axis Z can be transverse to the access port 231 at or near the center of the access port 231. The central axis Z can also define the central longitudinal axis of the conical shape of the cap 3, e.g., transverse to the radial direction of the conical shape.

The pressurized liquid 221 supplied by the fluid motion generator 5 can induce liquid circulation in the chamber 6 of the cap 3. For example, the fluid motion generator 5 (e.g., the inlet 61 and/or nozzle 210) can generate a swirling, rotational motion of influent liquid 222 about the central axis Z of the chamber, which can be transverse to (e.g., substantially perpendicular to in some arrangements) the X axis along which the liquid is introduced into the cap 3. In some arrangements, rotational or circulatory motion can also be induced about other directions, e.g., about an axis parallel to the direction of fluid introduction. As shown in FIG. 15, the influent liquid 222 can introduce rotational flow near and/or along walls 205 of the canal spaces 13 as the rotating liquid 222 enters the canal spaces 13.

In some embodiments, the pressurized liquid 221 can pass through the nozzle 210 and can emerge as a coherent, collimated liquid jet, which can act as a fluid motion generator and/or pressure wave generator, as explained above. In various embodiments of the nozzle 210, an orifice or opening in the nozzle may have a diameter d₁ at an inlet or a diameter d₂ at an outlet that may be in a range from about 5 microns to about 1000 microns. Other diameter ranges are possible. In various embodiments, one or both of the diameters d₁ or d₂ of the nozzle opening may be in a range from about 10 microns to about 100 microns, a range from about 100 microns to about 500 microns, or range from about 500 microns to about 1000 microns. In various other embodiments, one or both of the orifice diameters d₁ or d₂ may be in a range of about 40-80 microns, a range of about 45-70 microns, or a range of about 45-65 microns. In one embodiment, the orifice diameter d₁ is about 60 microns. The ratio of axial length L₁ to diameter d₁, the ratio of axial length L₂ to diameter d₂, or the ratio of total axial length L₁+L₂ to diameter d₁, d₂, or average diameter (d₁+d₂)/2 may, in various embodiments, be about 50:1, about 20:1, about 10:1, about 5:1, about 1:1, or less. In one embodiment, the axial length L₁ is about 500 microns. Additional examples of nozzles may be found in U.S. Patent Publication No. US 2011/0117517, which is incorporated by reference herein.

In some embodiments, the liquid 221 may comprise a stream of liquid that is not a jet, or that is not a circular jet. After entering the chamber 6, the liquid 221 can impact the side wall 220 of the cap 3. In some arrangements, the jet may impact an impingement surface before entering the chamber, e.g., a surface in the inlet path leading to chamber 6. The angle of the jet at the impact may be adjusted such that the impact leads to minimal loss of momentum. The fluid motion generator 5 can be angled such that, upon impingement of the liquid 221 against the wall 220, a rotating sheet of influent liquid 222 is generated in which the sheet of influent liquid 222 rotates in a swirling motion about the central axis Z and travels distally along the side wall 220 in the chamber 6 towards the opening 227. The rotating sheet of influent liquid 222 can continue downward along the inner walls 205 of the root canal(s) 13 towards the apical opening 15 of the tooth 10. The rotating liquid 222 can effectively and efficiently clean the entire root canal space 13. For example, the rapid, bulk fluid motion of the influent liquid 222 can interact with diseased matter in the root canal 13 and can dislodge or otherwise remove the diseased matter from the root canal 13.

Furthermore, in the embodiment shown in FIG. 15, when the liquid jet emerges from the nozzle 210, the jet can interact with treatment liquid in an interaction zone 230 near the interface between the nozzle 210 and the chamber 6. As explained herein, the liquid jet can pass through the liquid and can generate pressure waves 23 that propagate through the liquid in the chamber 6 and root canal 13 of the tooth 10. As shown in FIG. 15, and as explained above, the pressure waves 23 can propagate from the interaction zone 230 distally into the canal 13 of the tooth 10. The pressure waves 23 can comprise multiple frequencies that can cause liquid to flow into small spaces, cracks, and tubules of the tooth 10 to substantially clean the tooth 10. In some arrangements, the bulk flow of influent liquid 222 or large scale fluid motion may act to remove larger amounts of diseased material from relatively large spaces of the tooth, and the pressure waves 23 can flow into smaller spaces that may not be exposed to the bulk flow of liquid 222 or large scale fluid motion. The combination of rotating influent liquid 222 and pressure waves 23 can act to substantially clean the tooth, including large and small spaces of the tooth that may include different types and sizes of organic and inorganic matter.

It can be important to enable the influent liquid 222 to be removed from the treatment region to ensure that waste materials (e.g. dislodged debris, etc.) are irrigated from the tooth 10 and/or to enhance the fluid rotation at the treatment region. Accordingly, a fluid outlet 62 can be provided in and/or through the top wall 232 of the cap 3. The fluid outlet 62 can comprise a suction port 233 defining an opening between the chamber 6 and an outlet passage 209 (which may be one of the conduit(s) 7 described above) that conveys outgoing fluid to the waste system by way of a suction pump. The suction pump can apply suction to the outlet passage 209 and outlet 62 to draw fluids out of the chamber 6 and towards a reservoir outside the cap 3.

The outlet 62 and chamber 6 can be configured such that the influent liquid 222 turns back proximally at a return location to be drawn out of the chamber 6. The treatment liquid can turn back towards the cap 3 in an outgoing fluid path 224. The outgoing fluid path 224 may be different from the flow path or pattern of the influent liquid 222. For example, the returning or outgoing flow 224 path can comprise rotational (or semi-planar) flow near the center of the canal spaces and/or within the swirling influent flow path 222. In some embodiments, the outgoing flow 224 can comprise a spiral flow path that passes inside the rotating influent liquid 222. The induced outward flow 224 can be carried outside the treatment region to carry waste and other matter away from the treatment region (e.g., outside the canal 13 and tooth 10). Moreover, the suction provided by the outlet 62 and/or the rotating influent liquid 222 can provide a negative pressure at the apical opening 15 in which treatment liquid and/or waste is prevented from passing through the apical opening 15, which can reduce the risk of infection and/or pain to the patient. The outgoing liquid 224 can pass through the suction port 233 and can be drawn to the waste reservoir through the outlet line 209 by the suction pump. In addition, although not illustrated in FIG. 15, a vent assembly can be provided to enhance the removal of waste fluids from the system. For example, one or more vents can be provided through the cap 3 downstream of the suction port 233. In addition, in some embodiments, an auxiliary port can be provided on the cap 3. Examples of vent assemblies can be found in, e.g., U.S. Patent Publication No. 2012/0237893, which is incorporated by reference herein in its entirety. Furthermore, additional examples, of the system 2 shown in FIG. 15 may be found throughout U.S. Patent Publication No. US 2016/0095679, the entire contents of which are incorporated by reference herein in their entirety and for all purposes.

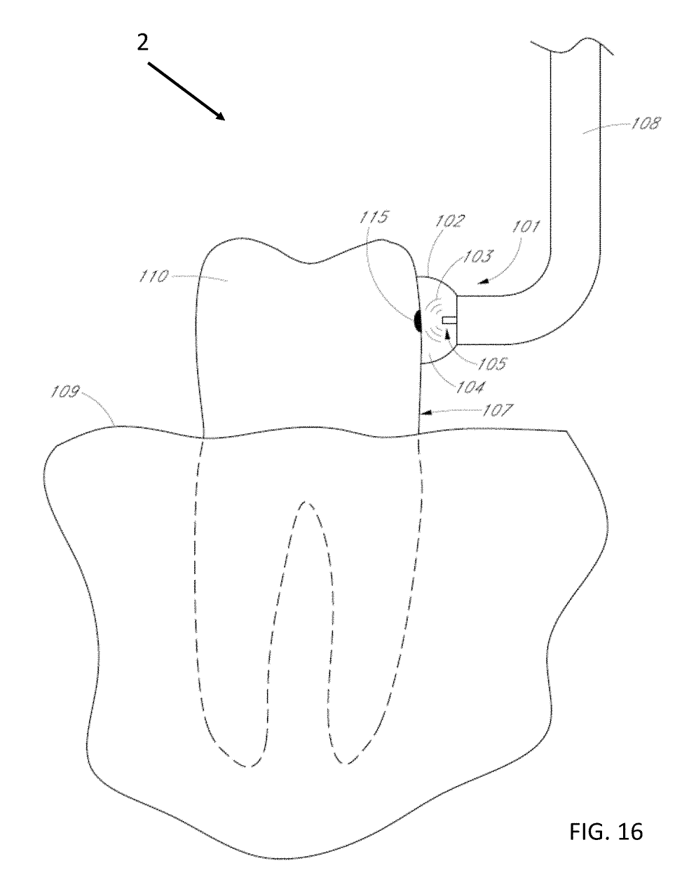

FIG. 16 schematically illustrates an example of a treatment system 2 for treating (e.g., cleaning) a carious region 115 on an exterior surface of a tooth 110. In particular, FIG. 16 is a schematic side cross-sectional view of the system 2 having a fluid platform 101 coupled to a treatment tooth 110 and that covers, or is positioned proximate to, a relatively small carious region 115 on the tooth 110. The carious region 115 illustrated in FIG. 16 may include a non-cavitated caries, e.g., a caries in which decay has progressed within the enamel, but not below the enamel into dentin. The carious region 115 of FIG. 16 may be formed in a side surface 107 of the tooth 110, such as a buccal or lingual surface of the tooth 110, as shown in FIG. 16. It should be appreciated, however, that the carious region 115 can be on other surfaces of the tooth 110. In the illustrated embodiment, the carious region 115 may be formed on the side surface 107 above a gum line 109 of the tooth 110.

The system 2 can include a handpiece 108, a fluid retainer or cap 102 configured to be attached to the tooth 110 over the carious region 115, and a pressure wave generator 105, which may generate broadband pressure waves as described above. The handpiece 108 can be provided to assist the clinician in positioning and coupling the cap 102 to the tooth 110. For example, the clinician can manipulate the handpiece 108 such that the cap 102 is disposed over and/or encloses the carious region 115. Furthermore, the handpiece 108 can be used by the clinician to position the cap 102 and the pressure wave generator 105 relative to the carious region 115 such that the pressure wave generator 105 is capable of generating sufficient acoustic energy to clean the carious region 115. For example, the clinician can use the handpiece 108 to position the cap 102 such that a distal portion of the pressure wave generator 105 is suitably spaced apart from and/or angled relative to the carious region 115 of the tooth 110. For example, the clinician, for various treatment reasons, may want to be able to position the pressure wave generator 105 at a particular distance from the carious region 115 and/or at a particular angle relative to the carious region 115 in order to achieve desirable treatment outcomes. In addition, as explained herein, the handpiece 108 can also, in some arrangements, include various inflow and outflow conduits to permit the transfer into and out of the cap 102 of suitable treatment fluids and/or waste fluids.

The cap 102 can be coupled to, or integrally formed with, the handpiece 108, e.g., at a distal portion of the handpiece 108. The cap 102 can be sized and shaped to retain fluid in a chamber 104 of the cap 102 when the cap 102 is attached or coupled to the tooth 110. In various embodiments, the chamber 104 of the fluid platform 101 can be at least partially filled with a liquid during treatment of the tooth 110. In some embodiments, for example, the chamber 104 can be substantially filled with liquid during treatment. For example, the chamber 104 can be filled above about 30% of the volume of the chamber 104, above about 50% of the volume of the chamber 104, above about 60% of the volume of the chamber 104, above about 75% of the volume of the chamber 104, above about 90% of the volume of the chamber 104, about 100% of the volume of the chamber 104, etc. The cap 102 can be configured to maintain a sealed liquid connection between the carious region 115 of the tooth 110 and the handpiece 108. For example, the cap 102 can be attached to the tooth 110 using an adhesive or sealant (not illustrated in FIG. 15). The adhesive or sealant can act to couple the cap 102 to the tooth 110 and/or to provide a liquid seal between the tooth 110 (e.g., the carious region 115) and the handpiece 108. In various embodiments, described below, treatment fluid can be introduced by way of one or more inlets from the handpiece 108 to the chamber 104 of the cap 102. In some embodiments, when the pressure wave generator 105 is a liquid jet, for example, the pressure wave generator 105 can introduce liquid into the chamber 104. In still other embodiments, a separate fluid introducer can be provided to introduce fluid into the chamber 104. The connection created between the cap 102 and the tooth 110 can be flexible such that the interface between the cap 102 and tooth 110 can accommodate movements in the handpiece 108 relative to the chamber 104, while maintaining the sealed connection. For example, the sealed connection between the cap 102 and the tooth 110 can allow the clinician to adequately position a distal portion of the pressure wave generator 105 relative to the carious region 115 of the tooth 110. The cap 70 can be formed from a sufficiently durable, biocompatible substance such as metal or plastic.

The pressure wave generator 105 can be coupled to the cap 102, and at least a portion of the pressure wave generator 105 can be disposed in the chamber 104. For example, a distal portion of the pressure wave generator 105 can be disposed in the chamber 104. The pressure wave generator 105 can be activated inside the chamber 104 of the cap 102 to clean the carious region 115 using generated acoustic waves 103. In some embodiments, the distal end portion of the pressure wave generator 105 can be submerged in the fluid inside the chamber 104. In other embodiments, the distal end portion of the pressure wave generator 105 can be disposed outside the fluid in the chamber 104.

The pressure wave generator 105 can generate the acoustic or pressure waves 103 within the liquid inside the chamber 104 in some embodiments. The pressure waves 103 can propagate throughout the liquid inside the enclosed volume formed by the chamber 104 and the cap 102, which can be sealed or attached to the tooth 110. Without being limited by theory, it is believed, although not required, that by applying sufficiently high-intensity pressure waves 103, acoustic cavitation may occur. The collapse of cavitation bubbles may induce, cause, or be involved in a number of processes such as, e.g., sonochemistry, tissue dissociation, tissue delamination, sonoporation, etc. The pressure wave field by itself may also be involved in one or more of the abovementioned processes. In some arrangements, the generation of pressure waves may or may not create or cause cavitation.

The pressure wave generator 105 can be any suitable pressure wave generator. For example, in some embodiments, the pressure wave generator 105 can include a liquid jet device. In particular, a coherent, collimated liquid jet can be formed by an orifice near a proximal portion of a guide tube. The jet can pass through a channel of the guide tube and can impact an impingement surface in some arrangements. The impact of the jet on the impingement surface can create the pressure waves 103 shown in FIG. 15. In some embodiments, the pressure waves 103 can propagate through the fluid that at least partially or substantially fills the chamber 104 of the cap 102. The pressure waves 103 can interact with the carious region 115 of the tooth to substantially remove decayed tooth matter, e.g., the caries. In some embodiments, the liquid that at least partially or substantially fills the chamber 104 can be a degassed liquid, which can improve cavitation and reduce the presence of gas bubbles inside the caries in some treatments. In other embodiments, the pressure wave generator 105 of FIG. 15 can include a mechanical pressure wave generator, an ultrasonic generator, an electromagnetic pressure wave generator (e.g., a laser), or a piezoelectric pressure wave generator. In still other embodiments, the pressure wave generator 105 can include a generator that transfers energy to particles within the treatment liquid that in turn creates pressure waves (e.g., photo-induced cavitation).

As explained herein, various conventional dental techniques may leave non-cavitated caries, such as the small caries shown in FIG. 15, untreated, or may only minimally treat the caries. Advantageously, the embodiment of FIG. 15 can detect and clean the non-cavitated, carious region 115 without harming the enamel or the underlying dentin. Furthermore, the system 2 of FIG. 15 can detect and clean such small carious regions 115 that may otherwise go undetected or untreated using conventional dental techniques. By detecting and cleaning even small caries (whether non-cavitated or cavitated), the system 2 disclosed herein can prevent further progression or worsening of the caries and can improve the overall health of the tooth 110. Additional details of the system 2 shown in FIG. 15 are disclosed throughout U.S. Patent Publication No. US 2015/0044632, the entire contents of which are incorporated by reference herein in their entirety and for all purposes.

A wide variety of other variations are possible. Components can be added, removed, and/or rearranged. For example, in some embodiments, the optical system does not include a thermally conductive housing or a thermoelectric controller. In some embodiments, the optical fibers can be oriented to direct light to the flow cell without the use of lenses or other optical elements. Other variations are also possible. Similarly, in any method or process disclosed herein, steps or operations can be added, removed, and/or rearranged.

Reference throughout this specification to “some embodiments,” “certain embodiments,” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment and may refer to one or more of the same or different embodiments. Furthermore, the particular features, structures or characteristics may be combined in any suitable manner, as would be apparent to one of ordinary skill in the art from this disclosure, in one or more embodiments.

As used in this application, the terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Similarly, it should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single foregoing disclosed embodiment.

Although the inventions presented herein have been disclosed in the context of certain preferred embodiments and examples, it will be understood by those skilled in the art that the inventions extend beyond the specifically disclosed embodiments to other alternative embodiments and/or uses of the inventions and obvious modifications and equivalents thereof. Thus, it is intended that the scope of the inventions herein disclosed should not be limited by the particular embodiments described above.

It will be appreciated that each of the processes, methods, and algorithms described herein and/or depicted in the figures may be embodied in, and fully or partially automated by, code modules executed by one or more physical computing systems, hardware computer processors, application-specific circuitry, and/or electronic hardware configured to execute specific and particular computer instructions. For example, computing systems can include general purpose computers (e.g., servers) programmed with specific computer instructions or special purpose computers, special purpose circuitry, and so forth. A code module may be compiled and linked into an executable program, installed in a dynamic link library, or may be written in an interpreted programming language. In some embodiments, particular operations and methods may be performed by circuitry that is specific to a given function.

Further, certain embodiments of the functionality of the present disclosure are sufficiently mathematically, computationally, or technically complex that application-specific hardware or one or more physical computing devices (utilizing appropriate specialized executable instructions) may be necessary to perform the functionality, for example, due to the volume or complexity of the calculations involved or to provide results substantially in real-time. For example, specifically programmed computer hardware may be necessary to generate a heat map based on one or more metrics representative of a characteristic of the examined portions of the teeth, overlap the heat map on one or more images of the examined portions of the teeth such that the heat map is registered with the pixels (or voxels) of examined portions of the teeth and/or display the heat map and the one or more images of the examined portions of the teeth in a commercially reasonable amount of time. As another example, specifically programmed computer hardware may be necessary to process the OCT images, the fluorescence images and/or the Raman maps of the examined portions of the teeth and extra information regarding condition of the examined portions of the teeth in a commercially reasonable amount of time.

Code modules or any type of data may be stored on any type of non-transitory computer-readable medium, such as physical computer storage including hard drives, solid state memory, random access memory (RAM), read only memory (ROM), optical disc, volatile or non-volatile storage, combinations of the same and/or the like. The methods and modules (or data) may also be transmitted as generated data signals (e.g., as part of a carrier wave or other analog or digital propagated signal) on a variety of computer-readable transmission mediums, including wireless-based and wired/cable-based mediums, and may take a variety of forms (e.g., as part of a single or multiplexed analog signal, or as multiple discrete digital packets or frames). The results of the disclosed processes or process steps may be stored, persistently or otherwise, in any type of non-transitory, tangible computer storage or may be communicated via a computer-readable transmission medium.

Any processes, blocks, states, steps, or functionalities in flow diagrams described herein and/or depicted in the attached figures should be understood as potentially representing code modules, segments, or portions of code which include one or more executable instructions for implementing specific functions (e.g., logical or arithmetical) or steps in the process. The various processes, blocks, states, steps, or functionalities can be combined, rearranged, added to, deleted from, modified, or otherwise changed from the illustrative examples provided herein. In some embodiments, additional or different computing systems or code modules may perform some or all of the functionalities described herein. The methods and processes described herein are also not limited to any particular sequence, and the blocks, steps, or states relating thereto can be performed in other sequences that are appropriate, for example, in serial, in parallel, or in some other manner. Moreover, the separation of various system components in the embodiments described herein is for illustrative purposes and should not be understood as requiring such separation in all embodiments. It should be understood that the described program components, methods, and systems can generally be integrated together in a single computer product or packaged into multiple computer products.

1. A dental system to optically examine a dental structure, the system comprising:
a mechanical assembly sized and shaped to be inserted into a mouth of a patient;
an optical assembly configured to provide an illumination beam to a portion of the dental structure and to collect light from the portion of the dental structure, the optical assembly mounted on the mechanical assembly; and
an electronic processing system configured to:
analyze the information recovered from the collected light; and
determine a characteristic representative of the portion of the dental structure based on the analysis.

2. The dental system of claim 1, wherein the mechanical assembly is configured to provide a controllable path for moving the optical assembly in the mouth of the patient.

3. The dental system of claim 1, wherein the mechanical assembly comprises a track or a rail.

4. The dental system of claim 1, wherein the optical assembly comprises an optical fiber.

5. The dental system of claim 1, wherein the optical assembly can be moved with respect to the dental structure.

6. The dental system of claim 1, wherein the optical assembly is a part of a fluorescence spectroscopy system.

7. The dental system of claim 1, wherein the optical assembly is a part of a Raman spectroscopy system.

8. The dental system of claim 1, wherein the optical assembly is a part of an optical coherence tomography (OCT) system.

9.-15. (canceled)

16. The dental system of claim 1, wherein the electronic processing system is configured to generate a heat map of the portion of the dental structure based on the determined characteristic.

17. A dental system to optically examine a dental structure, the system comprising:
a mechanical assembly sized and shaped to be inserted into a mouth of a patient;
an optical assembly configured to provide an illumination beam to a portion of the dental structure and to collect light from the portion of the dental structure, the optical assembly mounted on the mechanical assembly, the mechanical assembly configured to move the optical assembly relative to the dental structure to obtain a plurality of optical signals representative of a condition of the dental structure.

18. The dental system of claim 17, wherein the mechanical assembly comprises a track or a rail configured to facilitate movement of the optical assembly in the mouth of the patient.

19. The dental system of claim 17, wherein the optical assembly is laterally moved along an outer surface of the teeth to scan a plurality of locations on the outer surface of the teeth to detect caries.

20. The dental system of claim 17, wherein the optical assembly comprises an optical fiber configured to be inserted into a root canal or a periodontal pocket.

21.-24. (canceled)

25. The dental system of claim 17, wherein mechanical assembly is integrated in a mouthguard sized and shaped to be inserted into the mouth of the patient.

26. The dental system of claim 17, further comprising an electronic processing system configured to determine a characteristic representative of the condition of the dental structure and generate a heat map of the dental structure based on the determined characteristic.

27. The dental system of claim 17, further comprising a display device, wherein the electronic processing system is configured to display the heat map on the display device.

28. A dental system to optically examine a dental structure, the system comprising:
an optical assembly configured to provide an illumination beam to a portion of the dental structure and to collect light from the portion of the dental structure; and
an electronic processing system configured to:
analyze the information recovered from the collected light;
determine a characteristic representative of the portion of the dental structure based on the analysis; and
render the determined characteristic on a display device as a heat map, the heat map displaying different values of the determined characteristic with different indicia on the display device.

29. (canceled)

30. The dental system of claim 28, wherein the optical assembly comprises a beam-steering system configured to scan the illumination beam across the portion of the dental structure.

31. The dental system of claim 28, further comprising a movement assembly configured to move the optical assembly from a first position in a mouth of a patient to a second position in the mouth of the patient.

32. The dental system of claim 31, wherein the movement assembly comprises a track or a rail placed in the mouth of the patient.

33.-84. (canceled)
